(19) United States
(12) Patent Application Publication
(10) Pub. No.: US 2003/0077768 A1
(43) Pub. Date: Apr. 24, 2003

(54) Use of xylene monooxygenase for the oxidation of substituted polycyclic aromatic compounds

(76) Inventors: Michael G. Bramucci, Folsom, PA (US); Vasantha Nagarajan, Wilmington, DE (US); Stuart M. Thomas, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/214,059

(22) Filed: Aug. 7, 2002

Related U.S. Application Data
(60) Provisional application No. 60/311,486, filed on Aug. 10, 2001.

(51) Int. Cl. C12P 7/40

(52) U.S. Cl. 435/136

(57) ABSTRACT

The invention relates to a biocatalytic process for the oxidation of substituted polycyclic aromatic compounds to the corresponding carboxylic acids and related compounds. In a preferred embodiment the invention describes a method to produce 6-methyl-2-hydroxymethylnaphthalene, 6-methyl-2-naphthoic acid, 2,6-bis(hydroxymethyl)naphthalene and 2,6-naphthalenedicarboxylic acid from 2,6-dimethylnaphthalene. These compounds have been prepared by oxidizing 2,6-dimethylnaphthalene with a single recombinant microorganism containing the enzyme xylene monooxygenase.

This application claims the benefit of U.S. Provisional Application No. 60/311,486, filed Aug. 10, 2001.

### FIELD OF THE INVENTION

This invention relates to the field of molecular biology and microbiology. More specifically, this invention pertains to methods for the use of xylene monooxygenases comprising a XylA subunit and a XylM subunit for the oxidation of substituted polycyclic compounds and related ring structures. Of particular interest is the production of 2,6-naphthalenedicarboxylic acid and other oxidized derivatives of 2,6-dimethylnaphthalene by recombinant microorganisms containing xylene monooxygenase.

### BACKGROUND OF THE INVENTION

Oxidation of substituted polycyclic compounds is a common process used for the production of various chemical monomers. One specific application of this process is the production of 2,6-naphthalenedicarboxylic acid (2,6-NDC).

2,6-NDC is a monomer having utility in the production of polyesters which are commercially required in large quantities for fibers, films, paints, adhesives and beverage containers. A variety of chemical routes to 2,6-NDC are known, including catalytic oxidation of 2,6-dimethylnaphthalene (2,6-DMN). Additionally, methods for the oxidation of p-xylene to terephthalic acid can also be used for oxidation of 2,6-DMN to 2,6-NDC. For example, U.S. Pat. No. 2,833,816, (Amoco) teaches a process involving the oxidation of p-xylene with a molecular oxygen-containing gas in the liquid phase in a lower aliphatic monocarboxylic acid solvent in the presence of a heavy metal catalyst and a bromine compound to form terephthalic acid directly (U.S. Pat. No. 2,833,816). In specific embodiments, the reaction is catalyzed by Co and Mn in 95% acetic acid with a mixture of NH₄Br and tetrabromoethane as cocatalysts. The oxidation is carried out under severe conditions of high temperatures (109-205° C.) and pressures (15-30 bar). The rate of reaction is high and the yield of terephthalic acid based on p-xylene is as high as 95%. However, the reaction apparatus becomes heavily corroded owing mainly to the use of the bromine compound and the monocarboxylic acid solvent. Thus, ordinary stainless steel cannot be used to build the reaction apparatus, and expensive materials such as Hastelloy® or titanium are required. In addition, because the acid solvent is used in large quantity and the oxidation conditions are severe, combustion of the solvent itself cannot be avoided, and its loss is not negligible. Although it is possible to oxidize 2,6-DMN by these methods, they are expensive and generate waste streams containing environmental pollutants. An alternate route would be desirable.

In general, biological processes for production of chemicals are desirable for several reasons. One advantage is that the enzymes that catalyze biological reactions have substrate specificity. Accordingly, it is sometimes possible to use a starting material that contains a complex mixture of compounds to produce a specific chiral or structural isomer via a biological process. Another advantage is that biological processes proceed in a stepwise fashion under the control of enzymes. As a result, it is frequently possible to isolate the intermediates of a biological process more easily than the intermediates of an analogous chemical process. A third advantage is that biological processes are commonly perceived as being less harmful to the environment than chemical manufacturing processes. These advantages, among others, make it desirable to use 2,6-DMN as the starting material for manufacture of 2,6-NDC and partially oxidized derivatives of 2,6-DMN by means of a bioprocess.

Biological oxidation of methyl groups on aromatic rings, such as toluene and isomers of xylene is well known (U.S. Pat. No. 6,187,569; Dagley et al., Adv. Microbial Physiol. 6:1-46 (1971); Dutta et al., Appl. Environ. Microbiol. 64:1884-1889 (1998)). For example, bacteria that have the xyl genes for the Tol pathway sequentially oxidize the methyl group on toluene to afford benzyl alcohol, benzaldehyde and ultimately benzoic acid. The xyl genes located on the well-characterized Tol plasmid pWWO have been sequenced (Assinder et al., supra); Burlage et al., Appl. Environ. Microbiol. 55:1323-1328 (1989)). The xyl genes are organized into two operons. The upper pathway operon encodes the enzymes required for oxidation of toluene to benzoic acid. The lower pathway operon encodes enzymes that convert benzoic acid into intermediates of the tricarboxylic acid (TCA) cycle.

Xylene monooxygenase initiates metabolism of toluene and xylene by catalyzing hydroxylation of a methyl group on these compounds (Assinder et al., supra) Xylene monooxygenase has a NADH acceptor component (XylA) that transfers reducing equivalents to the hydroxylase component (XylM) (Suzuki et al., J. Bacteriol. 173:1690-1695 (1991)). This enzyme is encoded by xylA and xylM on plasmid pWWO (Assinder et al., supra). The cloned genes for the pWWO xylene monooxygenase have been expressed in Escherichia coli (Buhler et al., J. Biol. Chem. 275:10085-10092 (2000); Wubbolts et al., Enzyme Microb. Technol. 16:608-15 (1994); Harayama et al., J. Bacteriol. 167: 455-61 (1986)). The cloned xylene monooxygense oxidizes a variety of substituted toluenes to the corresponding benzyl alcohol derivatives. Although xylene monooxygenase is responsible for the first oxidation step of the Tol pathway and two distinct dehydrogenases are responsible for the next two oxidation steps in Pseudomonas putida (Harayama et al., supra), the cloned pWWO xylene monooxygenase has a relaxed substrate specificity and oxidizes benzyl alcohol and benzaldehyde to form benzoic acid (Buhler et al., supra).

In addition to the methyl groups on toluene and isomers of xylene, oxidation of the methyl groups on methyl- and dimethylnaphthalenes by bacteria is known (Grifoll et al., Appl. Environ. Microbiol. 61:3711-3723 (1995); Miyachi et al., Appl. Environ. Microbiol. 59:1504-1506 (1993); Dutta et al., supra). The genes responsible for oxidation of methyl- and dimethylnaphthalenes have not been identified. However, the metabolic pathway for oxidation of 2,6-DMN is similar to the Tol pathway for oxidation of p-xylene. Enzymes catalyze oxidation of one methyl group on 2,6-DMN to produce 2-hydroxymethyl-6-methylnaphthalene, 6-methyl-2-naphthaldehyde and 6-methyl-2-naphthoic acid. The primary pathway then involves cleaving the first ring (i.e, the ring that has the newly formed carboxyl group). Although the second methyl group typically is not oxidized until after the first ring has been metabolized, a small amout of 2,6-NDC may be formed as a dead end product (Dutta et al., supra).

In addition to these accounts methods have been described for using xylene monooxygenase to produce hydroxymethylated 5- or 6-atom aromatic heterocycles (U.S. Pat. No. 5,217,884), as well as for production of 2,6-NDC from 2,6-DMN using wildtype bacteria (U.S. Pat. No. 5,030,568).

Although the above-cited methods are useful for the oxidation of substituents on naphthalene and related ring structures, they involve multi-enzymatic processes for the oxidation of more than one substituent. The engineering of multi-enzyme processes into recombinant organisms is expensive and time consuming and requires regulation and expression of all the necessary enzymes.

The problem to be solved, therefore, is to provide an environmentally safe and economical method to oxidize substituted polycyclic compounds to industrially useful carboxylic acids and related compounds. Applicants have solved the stated problem through the discovery that xylene monooxygenases, having a XylM subunit and a XylA subunit are sufficient to oxidize multiple substituents on a polycyclic compound without the aid of additional enzyme intermediates. In particular Applicants have demonstrated that it is possible to produce 2,6-NDC and partially oxidized derivatives of 2,6-DMN using a single xylene monooxygenase species comprising the xylM and xylA genes cloned from Sphingomonas strain ASU1 and from the plasmid pWWO by expressing each enzyme separately in Escherichia coli in the presence of the appropriate substrate.

### SUMMARY OF THE INVENTION

The invention provides methods for the single step oxidation of methyl and other substituents on polycyclic compounds for the generation of polycyclic carboxylic acids and related compounds. The method uses the enzymatic activity of a xylene monooxygenase for the multiple oxidation of methyl and other alkyl groups on the ring structures. The method represents an advance over the art as heretofore all other xylene monooxygenases have only been shown to perform oxidation of only a single alkyl moiety on the ring.

The xylene monooxygenase of the present invention is sufficient to mediate the conversion of to 2,6-dimethylnaphthalene to 2,6-naphthalenedicarboxylic acid according to the following scheme: 2,6-dimethylnaphthalene→6-methyl-2-hydroxymethylnaphthalene→6-methyl-2-naphthaldehyde→6-methyl-2-naphthoic acid→6-hydroxymethyl-2-naphthoic acid→6-carboxy-2-naphthaldehyde→2,6-naphthalenedicarboxylic acid (FIG. 1).

Accordingly the invention provides a process for the oxidation of a substituted polycyclic aromatic substrate comprising:

(i) providing a recombinant microorganism comprising a DNA fragment encoding a xylene monooxygenase enzyme comprising an xylA subunit and an xylM subunit;

(ii) contacting the recombinant microorganism of step (i) with an aromatic substrate according to formula I

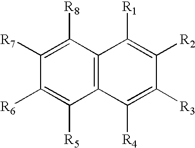

wherein R1-R8 are independently H, or CH3, or C₁ to C₂₀ substituted or unsubstituted alkyl or substituted or unsubstituted alkenyl or substituted or unsubstituted alkylidene, and wherein at least two of R1-R8 are present and are not H;

(iii) culturing the microorganism of step (ii) under conditions whereby anyone or all of R1-R8 is oxidized.

The process may be performed either in vivo using a recombinant organism expressing the xylene monooxygenase or in vitro with purified or partially purified enzyme.

In a specific embodiment the invention provides a process for the production of 2,6-naphthalenedicarboxylic acid comprising:

(i) providing a recombinant microorganism comprising a DNA fragment encoding a xylene monooxygenase enzyme comprising an xylA subunit and an xylM subunit;

(ii) contacting the recombinant microorganism of step (i) with an aromatic substrate selected from the group consisting of 2,6-dimethylnaphthalene, 6-methyl-2-hydroxymethylnaphthalene, 6-methyl-2-naphthoic acid, and 2,6-bis(hydroxymethyl)naphthalene; and

(iii) culturing the microorganism of step (ii) under conditions whereby 2,6-naphthalenedicarboxylic acid is produced.

In other specific embodiments the invention provides processes for the production of partially oxidized intermediates such as 6-methyl-2-hydroxymethylnaphthalene, 6-methyl-2-naphthoic acid, and 2,6-bis(hydroxymethyl)naphthalene comprising contacting the appropriate susbtituted polycylclic substrate with a xylene monooxygenase enzyme comprising a XylA subunit and an XylM subunit either in vivo or in vitro for the formation of the desired intermediate.

Additionally the invention provides a method for identifying a nucleic acid molecule encoding a xylene monooxygenase comprising:

(i) probing a genomic library with a portion of a nucleic acid molecule selected from the group consisting of SEQ ID NO:9, 11, 15, 17, 19, and 21;

(ii) identifying a DNA clone that hybridizes under conditions of 0.1×SSC, 0.1% SDS, 65° C. and washed with 2×SSC, 0.1% SDS followed by 0.1×SSC, 0.1% SDS with the nucleic acid molecule of (i); and

(iii) sequencing the genomic fragment that comprises the clone identified in step (ii),

wherein the sequenced genomic fragment encodes xylene monooxygenase.

Alternatively the invention provides a method for identifying a nucleic acid molecule encoding a xylene monooxygenase comprising:

(i) synthesizing at least one oligonucleotide primer corresponding to a portion of the sequence selected from the group consisting of SEQ ID NO:9, 11, 15, 17, 19, and 21; and

(ii) amplifying an insert present in a cloning vector using the oligonucleotide primer of step (i);

wherein the amplified insert encodes a xylene monooxygenase.

In another embodiment the invention provides an isolated nucleic acid molecule encoding the xylM subunit of a xylene monooxygenase selected from the group consisting of:

(i) an isolated nucleic acid molecule encoding the amino acid sequence as set forth in SEQ ID NO:10;

(ii) an isolated molecule encoding an amino acid sequence having at least 90% identity to the amino acid sequence as set forth in SEQ ID NO:10;

(iii) an isolated nucleic acid molecule that hybridizes with (i) or (ii) under the following hybridization conditions: 0.1×SSC, 0.1% SDS, 65° C. and washed with 2×SSC, 0.1% SDS followed by 0.1×SSC, 0.1% SDS; and

(iv) an isolated nucleic acid molecule that is completely complementary to (i), (ii) or (iii).

### BRIEF DESCRIPTION OF THE DRAWINGS AND THE SEQUENCES

FIG. 1 describes the enzymatic production of 2,6-Naphthalenedicarboxylic acid from 2,6-Dimethylnaphthalene.

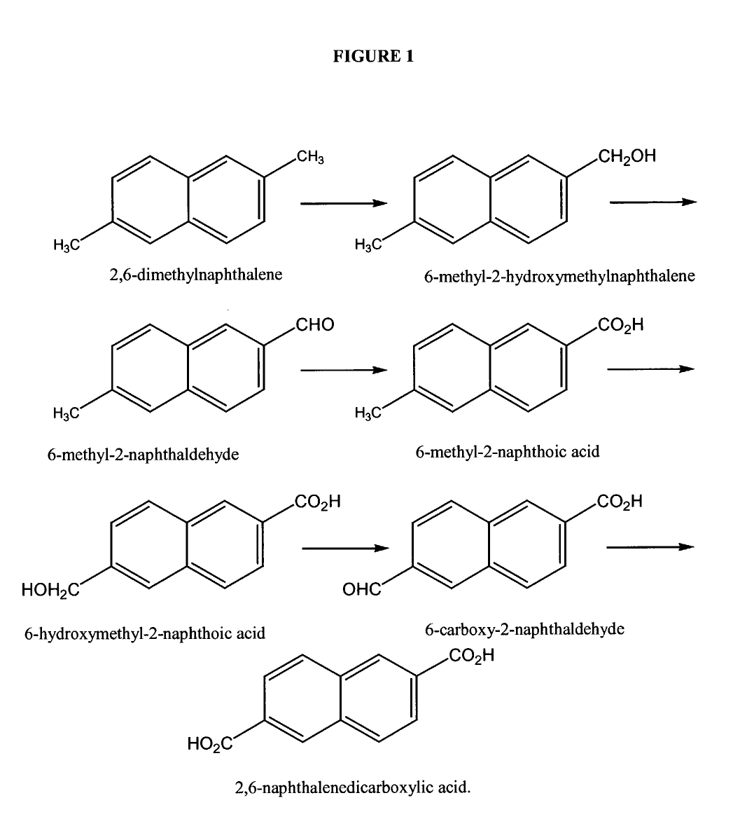

The invention can be more fully understood from the following detailed description and the accompanying sequence descriptions which form a part of this application.

The following sequence descriptions and sequence listings attached hereto comply with the rules governing nucleotide and/or amino acid sequence disclosures in patent applications as set forth in 37 C.F.R. §1.821-1.825 (“Requirements for Patent Applications Containing Nucleotide Sequences and/or Amino Acid Sequence Disclosures—The Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Adminstrative Instructions). The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NO:1 is primer xylAF1.

SEQ ID NO:2 is primer xylAR1.

SEQ ID NO:3 is primer JCR14.

SEQ ID NO:4 is primer JCR15.

SEQ ID NO:5 is 16S rRNA gene sequence from Sphingomonas strain ASU1.

SEQ ID NO:6 is Contig 12.5 which is 12,591 bp in length.

SEQ ID NO:7 is primer ASU1MAF1.

SEQ ID NO:8 is primer ASU1MAR1.

SEQ ID NO:9 is the nucleotide sequence for the Sphingomonas ASU1xylM gene.

SEQ ID NO:10 is amino acid sequence of the Sphingomonas ASU1 xylM.

SEQ ID NO:11 is the nucleotide sequence for the Sphingomonas ASU1 xylA gene.

SEQ ID NO:12 is amino acid sequence of Sphingomonas ASU1xylA.

SEQ ID NO:13 is primer WWOF1.

SEQ ID NO:14 is primer WWOR2.

SEQ ID NO:15 is the nucleotide sequence for the Pseudomonas pWWO xylM gene.

SEQ ID NO:16 is amino acid sequence of the Pseudomonas pWWO xylM.

SEQ ID NO:17 is the nucleotide sequence for the Pseudomonas pWWO xylA gene.

SEQ ID NO:18 is amino acid sequence of Pseudomonas pWWO xylA.

SEQ ID NO:19 is the nucleotide sequence for the Sphingomonas pNL1 xylM gene (GenBank Accession No. AF079317).

SEQ ID NO:20 is amino acid sequence of the Sphingomonas pNL1 xylM (GenBank Accession No. AF079317).

SEQ ID NO:21 is the nucleotide sequence for the Sphingomonas pNL1 xylA gene (GenBank Accession No. AF079317).

SEQ ID NO:22 is amino acid sequence of Sphingomonas pNL1 xylA (GenBank Accession No. AF079317).

### DETAILED DESCRIPTION OF THE INVENTION

The instant invention is a process for the oxidation of substituted polycylic compounds to the corresponding carboxylic acid and related compounds through the activity of a xylene monooxygenase. One specific application of the present method is the transformation of 2,6-DMN and partially oxidized compounds to 2,6-NDC using a single recombinant microorganism containing the enzyme xylene monooxgenase derived from Sphingomonas strain ASU1 or the enzyme xylene monooxgenase derived from the plasmid pWWO.

The instant invention is useful for the biological production of 2,6-NDC and other partially oxidized derivatives of 2,6-DMN which have utility in the production of polyesters needed in fibers, films, paints, adhesives and beverage containers. The instant invention advances the art of the synthesis of 2,6-NDC and other partially oxidized derivatives of 2,6-DMN as biological processes which are more cost effective and produce fewer environmentally harmful waste products.

In this disclosure, a number of terms and abbreviations are used. The following definitions are provided.

“2,6-Naphthalenedicarboxylic acid” is abbreviated 2,6-NDC.

“2,6-Dimethylnaphthalene” is abbreviated 2,6-DMN.

“6-Methyl-2-hydroxymethylnaphthalene” is abbreviated 6-M-2-HMN.

“6-Methyl-2-naphthoic acid” is abbreviated 6-M-2-NA.

“2,6-Bis(hydroxymethyl)naphthalene” is abbreviated 2,6-HMN.

“Open reading frame” is abbreviated ORF.

“Polymerase chain reaction” is abbreviated PCR.

As used herein, “ATCC” refers to the American Type Culture Collection International Depository located at 10801 University Boulevard, Manassas, Va. 20110-2209, U.S.A. The “ATCC No.” is the accession number to cultures on deposit with the ATCC.

The terms “biotransformation” and “bioconversion” will be used interchangeably and will refer to the process of enzymatic conversion of a compound to another form or compound. The process of bio-conversion or bio-transformation is typically carried out by a biocatalyst.

As used herein the term “biocatalyst” refers to a microorganism which contains an enzyme or enzymes capable of bioconversion of a specific compound or compounds.

The term “xylene monooxygenase” refers to an enzyme having the ability to oxidize methyl and other alkyl substitents on polycylic ring structures to the corresponding carboxylic acid.

The term “xylM” refers a DNA molecule encoding an iron containing hydroxylase subunit of a xylene monooxygenase.

The term “xylA” refers to a DNA molecule encoding a NADH binding electron transfer subunit of a xylene monooxygenase.

The term “substituted polycyclic aromatic substrate” refers to a compound having the general formula:

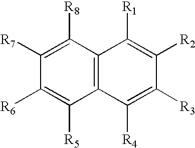

wherein R1-R8 are independently H, or CH3, or C₁ to C₂₀ substituted or unsubstituted alkyl or substituted or unsubstituted alkenyl or substituted or unsubstituted alkylidene, and wherein at least two of R1-R8 are present and are not H.

The term “alkyl” will mean a univalent group derived from alkanes by removal of a hydrogen atom from any carbon atom: CₙH₂ₙ₊₁—. The groups derived by removal of a hydrogen atom from a terminal carbon atom of unbranched alkanes form a subclass of normal alkyl (n-alkyl) groups: H[CH₂]ₙ—. The groups RCH₂—, R₂CH— (R not equal to H), and R₃C— (R not equal to H) are primary, secondary and tertiary alkyl groups respectively.

The term “alkenyl” will mean an acyclic branched or unbranched hydrocarbon having one carbon-carbon double bond and the general formula CₙH₂n. Acyclic branched or unbranched hydrocarbons having more than one double bond are alkadienes, alkatrienes, etc.

The term “alkylidene” will mean the divalent groups formed from alkanes by removal of two hydrogen atoms from the same carbon atom, the free valencies of which are part of a double bond (e.g. (CH₃)₂C=propan-2-ylidene).

The term, an “isolated nucleic acid fragment” or “isolated nucleic acid molecule” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding the xylene monooxygenase enzyme subunits as set forth in SEQ ID NOs:10, 12, 16, 18, 20 and 22. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

The present invention describes a process for the oxidation of substituted polycyclic aromatics via a xylene monooxygenase. A preferred process describes the production of 2,6-NDC and partially oxidized compounds involving the bioconversion of 2,6-DMN to 2,6-NDC using a single recombinant microorganism containing the enzyme xylene monooxgenase derived from Sphingomonas strain ASU1 or from the Pseudomonas plasmid pWWO. The genes for two subunits (xylM and xylA) of enzyme xylene monooxgenase have been cloned and expressed in a recombinant host for the bioconversion of 2,6-DMN and related compounds.

Two examples xylene monooxygenases suitable in the present invention have been isolated and demonstrated. One xylene monooxygenase was obtained from a bacterium that was isolated from activated sludge and that was typed as Sphingomonas sp. according to 16S rRNA sequence. The Sphingomonas ASU1 xylene monooxygenase XylM subunit is set forth in SEQ ID NO:10, encoded by the nucleic acid molecule as set forth is SEQ ID NO:9. The XylA subunit of the Sphingomonas ASU1 xylene monooxygenase is set forth in SEQ ID NO:12, encoded by the nucleic acid molecule as set forth in SEQ ID NO:11.

The other xylene monooxygenase of the instant invention is isolated from the plasmid pWWO contained in the bacterium Pseudomonas pudita strain ATCC 33015. The Pseudomonas xylene monooxygenase XylM subunit is set forth in SEQ ID NO:16, encoded by the nucleic acid molecule as set forth is SEQ ID NO:15. The XylA subunit of the Pseudomonas xylene monooxygenase is set forth in SEQ ID NO:18, encoded by the nucleic acid molecule as set forth in SEQ ID NO:17 (Assinder et al., supra).

As noted above, both the Spingomonas ASU1 xylene monooxygenase and the Pseudomonas xylene monooxygenase are comprised of two enzymatic subunits. One subunit is encoded by the xylA open reading frame and encodes an NADH binding electron transfer subunit. The other subunit is encoded by the xylM open reading frame which encodes an iron containing hydroxylase. The sequence of the Spingomonas XylM protein was compared with public databases using standard algorithms and was found to have 98% identity at the amino acid level with one other known gene.

Isolation Of Microorganisms Having xylene Monooxygenase Activity

Microorganisms having xylene monooxygenase activity may be isolated from a variety of sources. Suitable sources include industrial waste streams, soil from contaminated industrial sites and waste stream treatment facilities. The xylene monooxygenase containing microorganisms of the instant invention were isolated from activated sludge from a waste water treatment plant.

Samples suspected of containing a microorganism having xylene monooxygenase activity may be enriched by incubation in a suitable growth medium in combination with at least one substituted polycyclic aromatic organic substrate. Suitable aromatic organic substrates for use in the instant invention include, but are not limited to 2-methylnapthalene, 2,6-dimethylnaphthalene, 6-methyl-2-hydroxymethylnaphthalene, 6-methyl-2-naphthoic acid, 2,6-bis(hydroxymethyl)naphthalene, and 2,6-NDC.

Growth medium and techniques needed in the enrichment and screening of microorganisms are well known in the art and examples may be found in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)); or by Thomas D. Brock in Biotechnology: A Textbook of Industrial Microbiology, Second Edition, Sinauer Associates, Inc., Sunderland, Mass. (1989).

Characterization of the Xylene Monooxygenase Containing Microorganism:

One example of a xylene monooxygenase containing microorganism (strain ASU1) was identified as Sphingomonas sp. by analyzing the 16S ribosomal RNA (rRNA) gene sequence of the microorganism. The 16S rRNA gene sequence was amplified and cloned from strain ASU1 according to standard protocols (Maniatis, supra) and compared with sequences in public databases. The comparison revealed that the ASU1 16S rRNA sequence had significantly high homology to several strains of Sphingomonas.

Sphingomonas is included in the group Proteobacteria, of which Burkholderia, Alcaligenes, Pseudomonas, Sphingomonas, Novosphingobium, Pandoraea, Delftia and Comamonas are examples. The Proteobacteria form a physiologically diverse group of microorganisms and represent five subdivisions (α, β, γ, ε, δ) (Madigan et al., Brock Biology of Microorganisms, 8th edition, Prentice Hall, UpperSaddle River, N.J. (1997)). All five subdivisions of the Proteobacteria contain microorganisms that use organic compounds as sources of carbon and energy. Although the specific microorganism isolated was of the genus Sphingomonas, it is contemplated that other members of the Proteobacteria isolated according to the above method will be suitable, e.g. Pseudomonas (γ subdivision), because genes for metabolism of aromatic compounds are frequently located on plasmids and the plasmids are frequently capable of transferring between members of the Proteobacteria (Assinder et al., supra); Springael et al. Microbiol. 142:3283-3293 (1996)).

Thus it is contemplated that any xylene monooxygenase isolated from the group of bacteria, including but not limited to Burkholderia, Alcaligenes, Pseudomonas, Sphingomonas, Novosphingobium, Pandoraea, Delftia and Comamonas will be suitable in the present invention.

Identification of Xylene Monooxygenase Homologs

The present invention provides examples of xylene monooxygenase genes and gene products having the ability to bioconvert 2,6-DMN to 2,6-NDC. These include, but are not limited to the Sphingomonas ASU1 xylene monooxygenase (as defined by SEQ ID NO:9-12), the Pseudomonas xylene monooxygenase (strain ATCC 33015, Assinder et al., supra) as defined by SEQ ID NO:15-18) and the Sphingomonas plasmid pNL1 (GenBank Accession No. AF079317) xylene monooxygenase (as defined by SEQ ID NO:19-22). It will be appreciated that other xylene monooxygenase genes having similar substrate specificity may be identified and isolated on the basis of sequence dependent protocols.

Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g polymerase chain reaction (PCR)), Mullis et al., U.S. Pat. No. 4,683,202), ligase chain reaction (LCR), Tabor, S. et al., Proc. Acad. Sci. USA 82,1074, (1985)) or strand displacement amplification (SDA, Walker, et al., Proc. Natl. Acad. Sci. U.S.A., 89, 392, (1992)).

For example, genes encoding similar proteins or polypetides to the present xylene monooxygenases could be isolated directly by using all or a portion of the nucleic acid fragments set forth in SEQ ID NO:9, 11, 15, 17,19, and 21 or as DNA hybridization probes to screen libraries from any desired bacteria using methodology well known to those skilled in the art. Specific oligonucleotide probes based upon the instant nucleic acid sequences can be designed and synthesized by methods known in the art (Maniatis). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length DNA fragments under conditions of appropriate stringency.

Typically, in PCR-type primer directed amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art. (Thein and Wallace, “The use of oligonucleotide as specific hybridization probes in the Diagnosis of Genetic Disorders”, in Human Genetic Diseases: A Practical Approach, K. E. Davis Ed., (1986) pp. 33-50 IRL Press, Herndon, Va.); Rychlik, W. (1993) In White, B. A. (ed.), Methods in Molecular Biology, Vol.15, pages 31-39, PCR Protocols: Current Methods and Applications. Humania Press, Inc., Totowa, N.J.)

Generally PCR primers may be used to amplify longer nucleic acid fragments encoding homologous genes from DNA or RNA. However, the polymerase chain reaction may also be performed on a library of cloned nucleic acid fragments wherein the sequence of one primer is derived from the instant nucleic acid fragments. Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (GibcoBRL—Life Technologies, Rockville, Md.), specific 3′ or 5′ cDNA fragments can be isolated (Ohara et al., PNAS USA 86:5673 (1989); Loh et al., Science 243:217 (1989)).

Accordingly the invention provides a method for identifying a nucleic acid molecule encoding a xylene monooxygenase comprising: (a) synthesizing at least one oligonucleotide primer corresponding to a portion of the sequence selected from the group consisting of SEQ ID NO:9, 11,15, 17, 19, and 21 and (b) amplifying an insert present in a cloning vector using the oligonucleotide primer of step (a); wherein the amplified insert encodes a xylene monooxygenase

Alternatively the instant sequences may be employed as hybridization reagents for the identification of homologs. The basic components of a nucleic acid hybridization test include a probe, a sample suspected of containing the gene or gene fragment of interest, and a specific hybridization method. Probes of the present invention are typically single stranded nucleic acid sequences which are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions which will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen. Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Biotech, Milwaukee, Wis.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt./vol. glycine. Other additives may also be included, such as volume exclusion agents which include a variety of polar water-soluble or swellable agents, such as polyethylene glycol, anionic polymers such as polyacrylate or polymethylacrylate, and anionic saccharidic polymers, such as dextran sulfate.

Thus, the invention provides a method for identifying a nucleic acid molecule encoding a xylene monooxygenase comprising: (a) probing a genomic library with a portion of a nucleic acid molcule selected from the group consisting of SEQ ID NO:9, 11, 15, 17, 19, and 21; (b) identifying a DNA clone that hybridizes under conditions of 0.1×SSC, 0.1% SDS, 65° C. and washed with 2×SSC, 0.1% SDS followed by 0.1×SSC, 0.1% SDS with the nucleic acid molecule of (a); and (c) sequencing the genomic fragment that comprises the clone identified in step (b), wherein the sequenced genomic fragment encodes xylene monooxygenase.

Recombinant Expression

The genes and gene products of the present xylenes monooxygenase sequences may be introduced into microbial host cells. Preferred host cells for expression of the instant genes and nucleic acid molecules are microbial hosts that can be found broadly within the fungal or bacterial families and which grow over a wide range of temperature, pH values, and solvent tolerances. Because of transcription, translation and the protein biosynthetic apparatus is the same irrespective of the cellular feedstock, functional genes are expressed irrespective of carbon feedstock used to generate cellular biomass. Large scale microbial growth and functional gene expression may utilize a wide range of simple or complex carbohydrates, organic acids and alcohols, saturated hydrocarbons such as methane or carbon dioxide in the case of photosynthetic or chemoautotrophic hosts. However, the functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules that are added to the culture and are not typically considered nutrient or energy sources. Growth rate may also be an important regulatory factor in gene expression. Examples of suitable host strains include but are not limited to fungal or yeast species such as Aspergillus, Trichoderma, Saccharomyces, Pichia, Candida, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium, Klebsiella, Burkholderia, Sphingomonas, Novosphingobium, Paracoccus, Pandoraea, Delftia and Comamonas.

Microbial expression systems and expression vectors containing regulatory sequences that direct high level expression of foreign proteins are well known to those skilled in the art. Any of these could be used to construct chimeric genes for production of the any of the gene products of the instant sequences. These chimeric genes could then be introduced into appropriate microorganisms via transformation to provide high-level expression of the enzymes.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Initiation control regions or promoters, which are useful to drive expression of the instant ORF's in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

Once a suitable expression cassette is constructed comprising a xylene monooxygenase it may be used to transform a suitable host for use in the present method. Cassettes preferred in the present invention are those that contain both the xylM and the xylA subunits of the xylene monoxygenase wherein:

the xylM subunitis encoded by an isolated nucleic acid selected from the group consisting of:

(i) an isolated nucleic acid molecule encoding the amino acid sequence selected from the group consisting of SEQ ID NO:10, SEQ ID NO:16 and SEQ ID NO:20;

(ii) an isolated nucleic acid molecule having 95% identity to (i); and

(iii) an isolated nucleic acid molecule that is completely complementary to (i) or (ii)

and wherein:

xylA is encoded by an isolated nucleic acid selected from the group consisting of:

(i) an isolated nucleic acid molecule encoding the amino acid sequence selected from the group consisting of SEQ ID NO:12, SEQ ID NO:18, and SEQ ID NO:22;

(ii) an isolated nucleic acid molecule having 95% identity to (i); and

(iii) an isolated nucleic acid molecule that is completely complementary to (i) or (ii).

Process for the Production of 2,6-NDC and Intermediates:

The xylene monooxygenase of the instant invention may be used to oxidize a variety of substituted polycyclic aromatic compounds to the corresponding carboxylic acids and related compounds. Specifically the method of the present invention may be use to produce both 2,6-NDC and partially oxidized derivatives of 2,6-DMN.

Suitable substrates for the present reaction are defined by the formula:

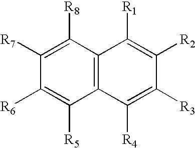

wherein R1-R8 are independently H, or CH3, or C₁ to C₂₀ substituted or unsubstituted alkyl or substituted or unsubstituted alkenyl or substituted or unsubstituted alkylidene, and wherein at least two of R1-R8 are present and are not H.

Where production of 2,6-NDC is desired substrates will include but are not limited to of 2,6-dimethylnaphthalene, 6-methyl-2-hydroxymethylnaphthalene, 6-methyl-2-naphthoic acid, and 2,6-bis(hydroxymethyl)naphthalene.

Where the production of 2,6-NDC is desired the recombinant microorganism containing xylene monooxygenase is contacted with 2,6-DMN in a suitable growth medium and the reaction medium is monitored for the production of 2,6-NDC. The instant process is also useful for the production of any of the intermediates of the 2,6-NDC biosynthetic pathway that may occur in the bioconversion of 2,6-DMN to 2,6-NDC.

Where commercial production of 2,6-NDC and other products is desired a variety of culture methodologies may be applied. For example, large scale production from a recombinant microbial host may be produced by both batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992), herein incorporated by reference.

Commercial production of 2,6-NDC and related compounds may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

### EXAMPLES

The instant invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Techniques suitable for use in the following Examples may be found in Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)); or by Thomas D. Brock in Biotechnology: A Textbook of Industrial Microbiology, Second Edition, Sinauer Associates, Inc., Sunderland, Mass. (1989). All reagents and materials used for the growth and maintenance of bacterial cells were obtained from Aldrich Chemicals (Milwaukee, Wis.), DIFCO Laboratories (Detroit, Ml), GibcoBRL—Life Technologies (Rockville, Md.) or Sigma Chemical Company (St. Louis, Mo.) unless otherwise specified.

The meaning of abbreviations is as follows: “h” means hour(s), “min” means minute(s), “sec” means second(s), “d” means day(s), “μL” means microliter, “mL” means milliliters, “L” means liters, “μm” means micrometer, “ppm” means parts per million (i.e., milligrams per liter).

Media:

Synthetic S12 medium was used to establish enrichment cultures. S12 medium contains the following: 10 mM ammonium sulfate, 50 mM potassium phosphate buffer (pH 7.0), 2 mM MgCl₂, 0.7 mM CaCl₂, 50 μM MnCl₂, 1 μM FeCl₃, 1 μM ZnCl₃, 1.72 μM CuSO₄, 2.53 μM CoCl₂, 2.42 μM Na₂MoO₂, 0.0001% FeSO₄ and 2 μM thiamine hydrochloride. S12 medium was routinely supplemented with yeast extract to a final concentration of 0.001%.

S12 agar was used to isolate bacteria from liquid enrichment cultures that grow on 2,6-DMN and to test isolates for growth with various sources of carbon and energy. S12 agar was prepared by adding 1.5% Noble agar (DIFCO) to S12 medium.

Since 2,6-DMN has low aqueous solubility, bacteria growing in S12 medium were supplied with 2,6-DMN by adding a few flakes of the compound directly to the culture. Bacteria growing on S12 agar were supplied with naphthalene, 2-methylnapthalene or 2,6-DMN by placing a few flakes of the compound on the interior of the petri dish lid. All petri dishes were sealed with parafilm and incubated with the lid on the bottom.

Luria-Bertani medium (1% of bacto-tryptone, 0.5% of bacto-yeast extract and 1% of NaCl) and/or standard M9 minimal medium were used to assay for oxidation of 2,6-DMN by Escherichia coli with cloned xylene monooxygenase. The M9 medium consisted of 42.3 mM Na₂HPO₄, 22.1 mM KH₂PO₄, 8.6 mM NaCl, 18.7 mM NH₄Cl, 2 mM MgSO₄, and 0.1 mM CaCl₂. Glycerol (0.4%) or 2,6-DMN was used as the carbon source.

Bacterial Strains and Plasmids:

Bacterial Sphingomonas strain ASU1 was isolated from activated sludge obtained from an industrial wastewater treatment facility. Pseudomonas pudita strain ATCC 33015 was obtained from the American Type Culture Collection (Manassas, Va.). Escherichia coli XL1-BlueMR and SuperCos 1 cosmid vector were purchased as part of the SuperCos 1 Cosmid Vector Kit (Stratagene, La Jolla, Calif.). Max Efficiency® competent cells of Escherichia coli DH5α was purchased from GibcoBRL—Life Technologies. Escherichia coli strain TOP10 and the plasmid vector pCR®2.1-TOPO™ used for cloning PCR products were purchased as a kit from Invitrogen—Life Technologes (Carlsbad, Calif.).

Construction of a Sphingomonas strain ASU1 Cosmid Library:

Sphingomonas strain ASU1 was grown in 25 mL LB medium for 16 h at 30° C. with shaking. Bacterial cells were centrifuged at 10,000 rpm for 10 min in a Sorvall® RC5C centrifuge using an SS34 rotor at 4° C. (Kendro Lab Products, Madison, Wis.). The supernatant was decanted and the cell pellet was gently resuspended in 2 mL of TE (10 mM Tris, 1 mM EDTA, pH 8). Lysozyme was added to a final concentration of 0.25 mg/mL. The suspension was incubated at 37° C. for 15 min. Sodium dodecyl sulfate was then added to a final concentration of 0.5% and proteinase K was added to a final concentration of 50 μg/mL. The suspension was incubated at 55° C. for 2 h. The suspension became clear and the clear lysate was extracted with an equal volume of phenol:chloroform:isoamyl alcohol (25:24:1). After centrifuging at 12,000 rpm for 20 min, the aqueous phase was carefully removed and transfered to a new tube. The aqueous phase was extracted with an equal volume of chloroform:isoamyl alcohol (24:1). After centrifuging at 12,000 rpm for 20 min, the aqueous phase was carefully removed and transfered to a new tube. The DNA was precipitated by adding 0.5 volumes of 7.5 M ammonium acetate and two volumes of absolute ethanol. The DNA was gently spooled with a sealed glass pasteur pipet. The DNA was gently washed with 70% ethanol and air dryed. The DNA was resuspended in 1 mL of TE. The DNA was treated with RnaseA (10 μg/mL final concentration) for 30 min at 37° C. The DNA was then extracted one time with phenol/chloroform, one time with chloroform and precipitated as described above. The DNA was resuspended in 1 mL of TE and stored at 4° C. The concentration and purity of DNA was determined spectrophotometrically by determining the ratio of the absorbance at 260 nm to the absorbance at 280 nm.

Chromosomal DNA was partially digested with Sau3A (Promega, Madison, Wis.) as outlined in the instruction manual for the SuperCos 1 Cosmid Vector Kit. DNA (30 μg) was digested with 0.8 units of Sau3A in a 50 μL reaction volume at 25° C. Aliquotes of 5 μL were withdrawn from the reaction tube at 5 min intervals until the reaction mixture was exhausted. Each aliquot was placed in a tube with 1 μL of gel loading buffer and 1 μL of 0.5M EDTA and was stored on ice until all of the aliquots had been collected. The aliquots were heated at 75° C. and analyzed on a 0.3% agarose gel to determine the extent of digestion. A decrease in size of chromosomal DNA corresponded to an increase in the length of reaction time. A preparative reaction was performed in which 30 μg of DNA was digested with 0.8 units of Sau3A in a 50 μL reaction volume at 25° C. for 30 min. The digestion was terminated by addition of 10 μL of 0.5M EDTA and heating the reaction for 10 min 75° C. The reaction was extracted once with an equal volume of phenol:chloroform:isoamyl alcohol and once with an equal volume of chloroform:isoamyl alcohol. The DNA was precipitated from the aqueous phase by adding 0.5 volumes of 7.5 M ammonium acetate and two volumes of absolute ethanol. The DNA was resuspended in 50 μL of water. The partially digested DNA was dephosphorylated with 1 unit calf intestinal alkaline phosphatase (CIAP) (GibcoBRL—Life Technologies) in 100 μL of reaction buffer supplied by the manufacturer. The reaction was incubated at 37° C. for 30 min. An additional 1 μL of CIAP was added and the reaction was incubated for another 30 min. The reaction was terminated by adding 600 μL of stop buffer (100 μL 1 M Tris pH 7.5, 20 μL 0.5M EDTA, 2 mL 1 M NaCl, 250 μL 20% SDS, 600 μL water) and incubating the reaction at 70° C. 10 min. The reaction was extracted once with an equal volume of phenol:chloroform:isoamyl alcohol and once with an equal volume of chloroform:isoamyl alcohol. The DNA was precipitated from the aqueous phase by adding 0.5 volumes of 7.5 M ammonium acetate and two volumes of absolute ethanol. The DNA was resuspended in 20 μL of TE.

The dephosphoylated ASU1 DNA was ligated to SuperCos 1 vector DNA which had been prepared according to the instructions supplied with the SuperCos 1 Cosmid Vector Kit. The ligated DNA was packaged into lamda phage coats using Gigapack® XL packaging extract as recommended by Stratagene and according to the manufacturer's instructions. The packaged ASU1 genomic DNA library contained a titer of 1.2×10³ colony forming units per μg of DNA as determined by infecting Escherichia coli XL1-Blue MR and plating the infected cells on LB agar with ampicillin (final concentration 50 μg/mL). Cosmid DNA was isolated from six randomly chosen Escherichia coli transformants and found to contain large inserts of DNA (25-40 kb).

Screening of a Strain ASU1 Cosmid Library for Xylene Monooxygenase Genes:

LB broth containing ampicillin (final concentration 50 μg/mL) was dipensed into the wells of microtiter plates (200 μL/well using Costar® #3595 with low evaporation lid (Corning Life Sciences, Acton, Mass.)). Each well was inoculated with one recombinant Escherichia coli colony. Each plate was covered with Air-Pore film (Qiagen, Valencia, Calif.), and the plates were incubated at 37° C. for 16 h on a shaking platform. These microtiter plates were designated “Culture Set #1”.

All of the cultures from Culture Set #1 were combined into 96 pools by mixing 10 μL aliquots from all of the wells that corresponded to each particular position on the microtiter plates, i.e., all of the wells in position Al were combined, all of the wells in position A2 were combined, etc. The pools were placed it in a new 96 well microtiter plate.

Each pool was diluted 1:10 and screened by PCR (2 μIL of pooled culture per 50 μL reaction) using a commercial kit according to the manufacturer's instructions (Perkin Elmer, Norwalk, Conn.)) with primer xylAF1 (CCGCACGATTGCAAGGT; SEQ ID NO:1) and primer xylAR1 (GGTGGGCCACACAGATA; SEQ ID NO:2). These primers were designed by aligning the XylA sequence encoded by Pseudomonas plasmid pWWO (GenBank® Accession No. P21394) with the XylA sequence encoded by the Sphingomonas plasmid pNL1 (GenBanke Accession No. AF079317) and identifying regions that were conserved in the two amino acid sequences. The pNL1 nucelotide sequence that corresponded to the conserved amino acid sequence was then used for primer design. PCR was performed in a Perkin Elmer GeneAmp® 9600. The samples were incubated for 1 min at 94° C. and then cycled 40 times at 94° C. for 1 min, 55° C. for 1 min, and 72° C. for 2 min. A 5 μL sample from each reaction was analyzed on a 0.8% agarose gel in TEA buffer using a Sunrise 96 Horizontal Gel Electrophoresis Apparatus (Invitrogen—Life Technologies, Catalog #11068-111). The gel ran for 1 h at 95 volts and was stained in TEA with ethidium bromide (8 μg/mL final concentration).

Pools that yielded a PCR product that was approximately 900 base pairs in length were deconvoluted by testing each individual culture fron Culture Set #1 that had been used to make the positive pool. LB broth containing ampicillin (final concentration 50 μg/mL) was dipensed into the wells of a microtiter plate (200 μL/well). Each well was inoculated with 10 μL of a culture from Culture Set #1. The microtiter plate was covered with Air-Pore film and incubated at 37° C. for 16 h on a shaking platform. Each culture was diluted 1:10. The diluted cultures were screened by PCR with primer xylAF1 (SEQ ID NO:1) and primer xylAR1 (SEQ ID NO:2), and the PCR products were analyzed by agaraose gel electrophoresis as described above.

Sequencing of a Cosmid Insert:

Cosmid DNA was subcloned for sequencing as follows. Clone E2/6 was used to prepare cosmid DNA from several mini-lysates according to the manufacturer's instructions supplied with the SuperCos 1 Cosmid Vector Kit. One library of subcloned cosmid DNA was constructed using DNA that had been fragmented by partial digestion with HaeIII (Promega). A second library of subcloned cosmid DNA was constructed using DNA that had been fragmented by nebulization.

Cosmid DNA (30 μL) was partially digested with 1 unit of HaeIII in a 50 μL reaction volume at 25° C. Aliquotes of 5 μL were withdrawn from the reaction tube at 5 min intervals until the reaction mixture was exhausted. Each aliquot was placed in a tube with 1 μL of gel loading buffer and 1 μL of 0.5 M EDTA and was stored on ice until all of the aliquots had been collected. The aliquots were heated at 75° C. and analyzed on a 0.8% agarose gel to determine the extent of digestion. A decrease in size of cosmid DNA corresponded to an increase in the length of reaction time. A preparative reaction was performed in the same way for 25 min. The reaction was stopped by addition of 10 1L of 0.5 M EDTA and incubation at 75° C. for 10 min. The fragments of partially digested DNA were separated according to size in a 0.8% low melting agarose gel in TEA buffer. DNA restriction fragments in the size range of 2 kb to 4 kb were excised from the gel and purified using a GeneClean® Kit according to the manufacturer's instructions (Qbiogene, Carlsbad, Calif.).

The cosmid DNA (45 μL) to be used for nebulization was treated with RNAse A (20 μg/mL final concentration; Sigma Chemical Co.) at 37° C. for 30 min. The DNA was purified by extraction with phenol/chloroform, extraction with chloroform and precipitation with ethanol. The DNA was resuspended in 50 μL of TE buffer. The DNA (50 μL) was diluted with 1 mL of water and was fragmented by forcing the solution through a nebulizer (IPI Medical Products, Chicago, Ill.; catalog number 4207) with filtered air (22 psi for 30 sec). The DNA fragments were concentrated by ethanol precipitation and separated according to size in a 0.8% low melting agarose gel in TEA buffer. DNA fragments in the size range of 2 kb to 4 kb were excised from the gel, purified using a GeneClean® Kit and resuspended in 40 μL of water. The ends of the DNA fragments were repaired in a 40 μL polishing reaction (4 μL 10× polynucleotide kinase buffer (Promega), 1 μL 10 mM ATP, 1 μL T4 Polymerase (6 units/μL; Promega), 1 μL Polynucleotide Kinase (6 units/μL; Promega), 30 μL nebulized DNA, 1.6 μL dNTPs (stock solution containing 2.5 nM of each dNTP), 1.4 μL water) that was incubated at 37° C. for 1 h. The reaction was terminated by incubation at 75° C. for 15 min. The polished DNA was purified using the GeneClean® Kit and resuspended in 20 μL of water.

Fragments of cosmid DNA produced by digestion with HaeII or by nebulization were ligated to SmaI cut plasmid pUC18 that was contained in a “Ready to Go” kit (Amersham Biosciences, Piscataway, N.J.). The ligated DNA was treated with the GeneClean® Kit according to the manufacturer's protocol and then electroporated into ElectroMAX™ DH10B™ Escherichia coli cells (Invitrogen—Life Technologies). Electroporation was performed with a Bio Rad Gene Pulser (Bio-Rad Laboratories, Hercules, Calif.) using settings of 2.5 kV, 25 μF and 200 z. The contents of the electroporation cuvette were tranferred to a 1.5 mL microcentrifuge tube and incubated at 37° C. for 1 h. Samples of the culture were spread on LB agar containing ampicillin (50 μg/mL) and X-gal (4 μg/mL of 5-bromo-4-chloro-3-indolyl-beta-D-galactopyranoside; Sigma Chemical Co., St. Louis, Mo.) and incubated at 37° C. for 16 h. One white colony was inoculated into each well of a 96 square-well plate (Beckman Coulter, Fullerton, Calif.) containing 1 mL of growth medium (LB containing 50 μg/mL ampicillin, 0.2% glucose and 20 mM Tris HCl, pH 7.5). The plates were incubated at 37° C. for 16 h on a shaking platform. Plasmid DNA was prepared from each culture using the Qiaprep 96 Turbo Miniprep Kit (Qiagen).

The plasmids were sequenced on an automated ABI sequencer (Applied Biosystems, Foster City, Calif.). The sequencing reactions were initiated with pUC18 universal and reverse primers. The resulting sequences were assembled using Sequencher 3.0 (Gene Codes Corp., Ann Arbor, Mich.).

HPLC:

HPLC system used was a Hewlett Packard 1100 series with a photo diode array detector and LC/MSD-ESI Negative ion. Column used was Hewlett Packard part #880975-902 Zorbax® SB-C18 (4.6×12.5 mm, 5 micron), purchased from Agilient Technologies (Foster City, Calif.). The column temperature was controlled at 30° C. Mobile phase was 0.02 mM ammonium acetate [20 mL 1 M ammonium acetate in 1800 mL water (solvent-2=S-2)] and acetonitrile (Solvent-1=S-1). The gradient used was 0-3 min 10% S-1 and 90% S-2, gradient was increased to 100% S-1 in 33 min, and again reduced to 10% S-1 and 90% S-2 in 3 min. Flow rate used for the mobile phase was 0.9 mL/min. 100 μL samples were injected, 230 nm wavelength was used for detection of the intermediates of 2,6-dimethylnaphthalene. Intermediates were prepared by as follows, as intermediates needed were not available in the market. Intermediates prepared were 6-methyl-2-napthoic acid, 6-methyl-2-hydroxymethylnaphthalene, and 2,6-Bis(hydroxymethyl)naphthalene. The retention time and diode array scans of the intermediates matched with the prepared standards. The mass spectrometer fragmentation pattern of the 6-methyl-2-napthoic acid in the sample matched that of the standards prepared.

GC/MS:

GC/MS was also used to analyze the samples. Samples for GC/MS analysis were extracted in equal volumes of ethyl acetate; the extract was dried with anhydrous magnesium sulfate and filtered. The extracts were evaporated to dryness under the gentile stream of nitrogen. Samples were then derivatized by BSTFA (bis(trimethylsilyl)trifluroacetamide silylation reagent (Supelco, Bellefonte, Pa.)) before injecting onto GC column. Instruments used were Finnigan SSQ® 7000 (Thermo Finnigan, San Jose, Calif.) with Hewlett-Packard 5980 II plus GC or Hewlett-Packard 5970 MSD with 5980; both mass spectrometers are single-stage quadrupole instruments. GC samples were run on an MDN-5S column, 1 μL splitless injection and 13-min delay time before turning mass spectrometer on. GC conditions for column temperature gradient was 50° C. for 5 min ramped at 10° C./min to 300° C., held for 5 min.

Identification of 2,6-DMN Metabolites:

The conversion of 2,6-DMN to 2,6-NDC was monitored by reverse phase HPLC. Culture supernatants were passed through 0.2 μm filters (Gelman Acrodisc® CR PFTE (Gelman/Pall Life Sciences, Ann Arbor, Mich.) or Millipore Corp. Millex®-gs(Bedford, Mass.)) prior to analysis. Analyses were performed on either a Hewlett Packard HPLC model 1050 equipped with a Milton Roy LDC single wavelength detector set at 214 nm or a Hewlett Packard HPLC model 1090 equipped with a diode array UV-visible detector set at 254 nm (primary wavelength), 230 nm (secondary wavelength) and 450 nm as background reference. Samples (10 μL) were injected onto a Zorbax® C8 column (2.1 mm×15 cm). The mobile phase consisted of (A) H₂O containing 2 mL phosphoric acid/L and (B) acetonitrile. Gradients were as follows: a) 0 min to 25 min (B) increased from 10% to 25%, b) (B) increased to 95% over the next 12 min, c) (B) was held at 95% for 3 min, and d) (B) decreased to 10% in 1 min. All calibrations and data analysis was done using Hewlett Packard's Chemstation Software. Preparative HPLC for peak collection was run on instrument 11 with either a Zorbax® RXC8 9.4 mm×25 cm with a 50-250 μL injection volume. The mobile phase consisted of (A) H₂O containing 2 mL phosphoric acid/L or 2 mL acetic acid and (B) acetonitrile. For peak collection, samples were run in 2 mL acetic acid/1L of Milli-Q® water mobile phase (Millipore Corp.). Peaks of interest were collected into 20 mL glass vials. Samples were then subsequently concentrated in a Savant Speed Vac® (Thermo Savant, Holbrook, N.Y.).

Synthesis of 6-Methyl-2-naphthoic acid, 6-Methyl-2-hydroxymethylnaphthalene and 2,6-Bis(hydroxymethyl)naphthalene for Use as Analytical Standards:

6-Methyl-2-naphthoic acid:

A solution of potassium hypochlorite was prepared by dissolving 8.16 g of calcium hypochlorite, containing 65% of active ingredient, in 31.5 mL of water, followed by adding a warm solution of 5.73 g of potassium carbonate and 1.77 g of potassium hydroxide in 16.5 mL of water, shaking well, filtering and washing the precipitate one time with distilled water into the original filtrate. The solution of potassium hypochlorite was heated to 55° C. with stirring and 3.0 g (16.28 mmol), of 6-methyl-2-acetonaphthone (Aldrich Chemical Co.), was added. The temperature was kept at 60-65° C. as the solution was stirred overnight. The excess hypochlorite was destroyed by adding a solution of 3.0 g (17.23 mmol), of sodium hydrosulfite (Aldrich Chemical Co.) in 15 mL of water. The solution was filtered while hot. After cooling to room temperature, the reaction mixture was transferred to a 150 mL beaker and carefully acidified with 7.5 mL of concentrated hydrochloric acid. The crude product was collected on a Buchner funnel, washed with water and dried under vacuum. The crude product was crystallized from 100 mL of 95% alcohol and washed with CHCl₃ giving 1.91 g of 6-methyl-2-naphthoic acid (63% yield), mp 226-228° C. (lit. 225-227° C., J. Chem. Soc., 1784 (1932)). The ¹H NMR (DMSO-d₆) and IR (KBR) are consistent with the proposed structure. Elemental analysis: Found: C, 77.11; H, 5.41: Calc'd: C, 77.40, H, 5.41.

6-Methyl-2-hydroxymethylnaphthalene:

To 0.5 g of the 6-methyl-2-naphthoic acid in 140 mL of dry tetrahydrofuran under nitrogen was added 0.238 g (6.25 mmol) of lithium aluminum hydride portion-wise. The mixture was heated at reflux for 6 h and, after cooling to room temperature, the excess lithium aluminum hydride was decomposed by the portion-wise addition of saturated ammonium chloride solution. The slurry was filtered and the filtrate was concentrated on a rotatory evaporator. The crude product was purified by silica gel chromatography using chloroform as the eluent yielding 0.417 g of 6-methyl-2-hydroxymethylnaphthalene (90% yield) as a white, crystalline solid. The ¹H NMR and IR are as expected for the desired product. ¹H NMR (CDCl₃.): s, 2.45 (3H); s, 4.78 (2H); m, 7.50 (6H).

2,6-Bis(carbomethoxy)naphthalene:

1.03 g (4.76 mmol) of 2,6-naphthalenedicarboxylic acid (Aldrich Chemical Co.) was dissolved in 43 mL H₂SO₄ and 150 mL of dry methanol was added cautiously. The reaction mixture was heated at reflux for 1 h. After cooling to room temperature, the mixture was neutralized cautiously with saturated aqueous Na₂CO₃ solution and filtered to give the crude product as a white solid which was recrystalized from ethanol to afford 0.9387 g of 2,6,-bis(carbomethoxy)naphthalene (93% yield) which was pure by ¹H NMR analysis. ¹H NMR (CDCl₃.): S, 4.04 (6H); q, 8.00, (4H); s, 8.65, (2H).

2,6-Bis(hydroxymethyl)naphthalene:

To 0.288 g of 2,6-bis(carbomethoxy)naphthalene (1.36 mmol) in 150 mL of tetrahydrofuran under nitrogen was added 0.290 g (7.64 mmol) of lithium aluminum hydride portion-wise. The mixture was heated at reflux for 8 h and the excess lithium aluminum hydride was decomposed by addition of saturated ammonium chloride solution. The slurry was filtered, the solid was rinsed with fresh tetrahydrofuran into the original filtrate and the filtrate was concentrated to dryness on a rotary evaporator yielding 0.235 g of crude product as a white solid which was recrystalized from EtOH/CH₂Cl₂ to afford 120 mg of 2,6-bis(hydroxymethyl)naphthalene (47% yield), mp 170-172° C. (lit. 170-170.5° C.; U.S. Pat. No. 3,288,823 (1966)). The ¹H NMR (DMSO-d₆) and IR (KBr) were clean and as expected. ¹H NMR (DMDO-d6): s, 4.90 (4H); q, 7.80 (4H); s, 8.08 (2H).

### EXAMPLE 1

### Isolation and Characterization of Sphingomonas Strain ASU1

This Example describes the isolation of strain ASU1 on the basis of being able to grow on 2,6-DMN as the sole source of carbon and energy. The ability of strain ASU1 to grow on various substrates indicated that strain ASU1 utilized the TOL pathway or a similar pathway to degrade 2,6-DMN. Analysis of a 16S rRNA gene sequence indicated that strain ASU1 was related to a member of the α-Proteobacteria belonging to the genus Sphingomonas.

Bacteria that grow on 2,6-DMN were isolated from an enrichment culture. The enrichment culture was established by inoculating 0.1 mL of activated sludge into 10 mL of S12 medium in a 125 mL screw cap Erlenmeyer flask. The activated sludge was obtained from a DuPont wastewater treatment facility. The enrichment culture was supplemented with adding yeast extract (0.001% final concentration) by adding a few flakes of 2,6-DMN directly to the culture medium. The enrichment culture was incubated at 28° C. with reciprocal shaking. The culture was diluted every 4 to 7 d by replacing 9 mL of the culture with the same volume of S12 medium with 0.001% yeast extract and a few additional flakes of 2,6-DMN. Bacteria that utilized 2,6-DMN as a sole source of carbon and energy were isolated by spreading samples of the enrichment culture onto S12 agar. 2,6-DMN was placed on the interior of each Petri dish lid. The Petri dishes were sealed with parafilm and incubated upside down at 28° C. Representative bacterial colonies were then tested for the ability to use 2,6-DMN as a sole source of carbon and energy. Colonies were transferred from the S12 agar plates to S12 agar plates and supplied with 2,6-DMN on the interior of each Petri dish lid. The Petri dishes were sealed with parafilm and incubated upside down at 28° C. The isolates that utilized 2,6-DMN for growth were then tested for growth on S12 agar plates containing other aromatic compounds.

The 16S rRNA genes of strain ASU1 were amplified by PCR and analyzed as follows. ASU1 was grown on LB agar (Sigma Chemical Co.). Several colonies were suspended in 100 mL of water that had been passed through a 0.22μ filter. The cell suspension was frozen at −20° C. for 30 min, thawed at room temperature and then heated to 90° C. for 10 min. Debris was removed by centrifugation at 14,000 RPM for 1 min in a Sorvall® MC 12V microfuge. The 16S rRNA gene sequences in the supernatant were amplified by PCR by using a commercial kit according to the manufacturer's instructions (Perkin Elmer) with primers JCR14 (ACGGGCGGTGTGTAC; SEQ ID NO:3) and JCR15 (GCCAGCAGCCGCGGTA; SEQ ID NO:4). PCR was performed in a Perkin Elmer GeneAmp® 9600. The samples were incubated for 5 min at 94° C. and then cycled 35 times at 94° C. for 30 sec, 55° C. for 1 min, and 72° C. for 1 min. The amplified 16S rRNA genes were purified using a commercial kit according to the manufacturer's instructions (QIAquick PCR Purification Kit, Qiagen, Inc.) and sequenced on an automated ABI sequencer. The sequencing reactions were initiated with primers JCR14 (SEQ ID NO:3) and JCR15 (SEQ ID NO:4). The 16S rRNA gene sequence of each isolate was used as the query sequence for a BLAST search (Altschul, et al., Nucleic Acids Res. 25:3389-3402(1997)) of GenBank® for similar sequences.

A 16S rRNA gene of strain ASU1 was sequenced and compared to other 16S rRNA sequences in the GenBank® sequence database. The 16S rRNA gene sequence from strain ASU1 (SEQ ID NO:5) had significantly high homology to several 16S rRNA gene sequences of α-Proteobacteria belonging to the genus Sphingomonas. The ASU1 sequence had the highest homology (99.6% identity) to the 16SrRNA gene sequence isolated from Sphingomonas strain MBIC3020 (GenBank° Assession No. AB025279.1).

The data in Table 1 indicated strain ASU1 was able to grow on 2,6-DMN and several other methylated aromatic compounds. However, strain ASU1 was unable to utilize benzene.

TABLE 1
Summary of Carbon Source Utilization for Strain ASU1

| Carbon Source | Growth on Carbon Source |
|---|---|
| benzene | − |
| toluene | + |
| p-xylene | + |
| naphthalene | + |
| 2-methylnaphthalene | + |
| 2,6-dimethylnaphthalene | + |

### EXAMPLE 2

### Cloning of the Genes for Xylene Monooxygenase from Sphingomonas Strain ASU1

This Example describes the cloning of xylene monooxygenase genes (xylM and xylA) from Sphingomonas strain ASU1. The xylM and xylA genes from strain ASU1 were homologous to the xylene monooxygenase genes found on plasmid pNL1 (Romine et al., J. Bacteriol. 181:1585-602 (1999)). The ASU1 xylM and xylA genes were expressed in Escherichia coli.

Two positive clones (E2/6 and G9/6) were identified among about 700 cosmid clones that contained ASU1 DNA and were screened by PCR using primers xylAF1 (SEQ ID NO:1) and xylAR1 (SEQ ID NO:2). Both of the clones contained inserts of 35-40 kb. A library of subclones was constructed from cosmid E2/6 using pUC18. The pUC18 subclones were sequenced with pUC18 universal and reverse primers. The sequences were assembled using Sequencher 3.0. One of the contigs was 12,591 bp in length. This sequence (Contig 12.5; SEQ ID NO:6) was analyzed by conducting BLAST (Basic Local Alignment Search Tool; Altschul et al., J. Mol. Biol. 215:403-410 (1993)); see also www.ncbi.nlm.nih.gov/BLAST) searches for similarity to sequences contained in the GenBank® databases. Contig 12.5 (SEQ ID NO:6) was compared to all publicly available DNA sequences contained in the GenBank® nucleotide database using the BLASTN algorithm provided by the National Center for Biotechnology Information (NCBI). Large portions of Contig 12.5 (SEQ ID NO:6) were found to have homology with plasmid pNL1 (GenBank® Accession No. AF079317; Table 2). Contig 12.5 (SEQ ID NO:6) was analyzed for ORFs by using the BLASTX algorithm (Gish, W. and States, D. J. Nature Genetics 3:266-272 (1993)), provided by the NCBI, which was used to detect ORFs in Contig 12.5 (SEQ ID NO:6) by translating Contig 12.5 (SEQ ID NO:6) in all 6 reading frames and comparing the translation products to all publicly available protein sequences contained in the GenBank® “nr” database. Region 2 of Contig 12.5 (SEQ ID NO:6) contained two ORFs that were homologous to the xylA gene and xylM gene on plasmid pNL1. The sequence comparisons based on the BLASTX analysis against the protein database are given in Table 3.

A fragment of ASU1 DNA that contained xylM and xylA was cloned into a small, multicopy plasmid. Primers ASU1 MAF1 (TAACTAAGGAGAAATCATATGGACGGACTGCG; SEQ ID NO:7) and ASU1MAR1 (GGATCCCGGGTCTTTTTTTACGTGCGATTGCTGCG; SEQ ID NO:8) were used to amplify a 2.3 kb fragment by PCR by using a commercial kit according to the manufacturer's instructions (Perkin Elmer). PCR was performed in a Perkin Elmer GeneAmp® 9600 using DNA from Sphingomonas strain ASU1. The samples were incubated for 1 min at 94° C. and then cycled 40 times at 94° C. for 1 min, 55° C. for 1 min, and 72° C. for 2 min. After the last cycle, the samples were incubated at 72° C. for an additional 10 min. The amplified DNA was purified using a commercial kit according to the manufacturer's instructions (QIAquick PCR Purification Kit, Qiagen, Inc.). The purified DNA was ligated into pCR®2.1 TOPO™ and transformed into Escherichia coli TOP10 using a TOPO™ TA Cloning® Kit according to the manufacturer's instrutions (Invitrogen—Life Technologies). The transformed cells were spread on LB agar containing 50 μg/mL of ampicillin at 37° C. for 24 h. The plates were then incubated at room temperature (approximately 25° C.) another 1 to 2 d until some colonies turned blue.

The formation of blue colonies was due to monooxygenase mediated conversion of indole to indigo (Keil et al., J. Bacteriol. 169:764-770 (1987); O'Connor et al., Appl. Environ. Microbiol. 63:4287-4291 (1997)). Formation of indigo indicated that a clone contained the ASU1 xylM and xylA genes and that a functional xylene monooxygenase was being expressed from the cloned genes.

TABLE 2
BLASTN Analysis of Contig 12.5 (SEQ ID NO:6)
Position (bp)

| Region | Contig 12.5 | pNL1 | % identity |
|---|---|---|---|
| 1 | 632-6417 | 133,938- | 97 |
| | | 139,727 | |
| 2 | 8554-12,591 | 141,896- | 92 |
| | | 145,937 | |

TABLE 3

| | Gene | | SEQ ID | SEQ ID | % | % | |
|---|---|---|---|---|---|---|---|
| ORF | Name | Similarity Identified | base | Peptide | Identity^{a} | Similarity^{b} | E-value^{c} |
| 1 | xy/M | xy/M from pNL1, | 9 | 10 | 98.4 | 98.9 | 0.0 |
| | | GenBank ® Accession No. | | | | | |
| | | AF079317 | | | | | |
| 2 | xy/A | xy/A from pNL1, | 11 | 12 | 99.4 | 100 | 0.0 |
| | | GenBank ® Accession No. | | | | | |
| | | AF079317 | | | | | |

% ^{a}Identity is defined as percentage of amino acids that are identical between the two proteins.
% ^{b}Similarity is defined as percentage of amino acids that are identical or conserved between the two proteins.
^{c}Expect value. The Expect value estimates the statistical significance of the match, specifying the number of matches, with a given score, that are expected in a search of a database of this size absolutely by chance.

### EXAMPLE 3

### Cloning of the Genes for Xylene Monooxygenase from Pseudomonas putida

This Example describes the cloning of the genes for xylene monooxygenase from Pseudomonas putida.

A fragment of pWWO DNA that contained xylM and xylA was cloned into a small, multicopy plasmid. Primers WWOF1 (TMGTAGGTGGATATATGGACAC; SEQ ID NO:13) and WWOR2 (GGATCCCTAGACTATGCATCGAACCAC; SEQ ID NO:14) were used to amplify a 2.4 kb fragment by PCR by using a commercial kit according to the manufacturer's instructions (Perkin Elmer). PCR was performed in a Perkin Elmer GeneAmp® 9600 using DNA from Pseudomonas pudita strain ATCC 33015. The samples were incubated for 1 min at 94° C. and then cycled 40 times at 94° C. for 1 min, 55° C. for 1 min, and 72° C. for 2 min. After the last cycle, the samples were incubated at 72° C. for an additional 10 min. The amplified DNA was purified using a commercial kit according to the manufacturer's instructions (QIAquick PCR Purification Kit, Qiagen, Inc.). The purified DNA was ligated into pCR®2.1-TOPO™ and transformed into Escherichia coli TOP10 using a TOPO™ TA Cloning® Kit according to the manufacturer's instrutions (Invitrogen—Life Technologies). The transformed cells were spread on LB agar containing 50 μg/mL of ampicillin at 37° C. for 24 h. The plates were then incubated at room temperature (approximately 25° C.) another 1 to 2 d until some colonies turned blue.

The formation of blue colonies was due to monooxygenase mediated conversion of indole to indigo (Keil et al., J. Bacteriol. 169:764-770 (1987); O'Connor et al., Appl. Environ. Microbiol. 63:4287-4291 (1997)). Formation of indigo indicated that a clone contained the pWWO xylM and xylA genes and that a functional xylene monooxygenase was being expressed from the cloned genes.

### EXAMPLE 4

### Oxidation of 2,6-DMN by Escherichia coli Recombinants with Cloned ASU1 Xylene Monooxygenase Genes or pWWO Xylene Monooxygenase Genes

Example 4 demonstrated that Escherichia coli recombinants with xylene monooxygenase genes cloned from Sphingomonas strain ASU1 (Clone 4a) or cloned from plasmid pWWO (Clone 6f) oxidized 2,6-DMN to form 6-methyl-2-hydroxymethylnaphthalene (6-M-2-HMN) and 6-methyl-2-naphthoic acid (6-M-2-NA). In addition, 2,6-bis(hydroxymethyl)naphthalene (2,6-HMN) was detected in the culture supernatant of Clone 6f, and 2,6-NDC was detected in the culture supernatant of Clone 4a. These results indicate that 2,6-DMN is a substrate for the ASU1 xylene monooxygenase and the pWWO xylene monooxygenase. Furthermore, these monooxygenases are able to oxidize both methyl groups of 2,6-DMN.

Escherichia coli strain TOP10 (pCR®2.1-TOPO™) and Escherichia coli clones expressing xylene monooxygenase (Clone 4a and Clone 6F) were inoculated into 500 mL Erlenmyer flasks containing 50 mL of LB that was supplemented with 50 μg/mL ampicillin. The cultures were incubated 25 h at 37° C. with reciprocal shaking. The cells were harvested from each culture by centrifugation and resuspended to a final optical density at 600 nm (OD₆₀₀) of 0.8 in M9 medium that was supplemented with 50 μg/mL ampicillin, 0.4% glycerol, 0.4% casamino acids (DIFCO) and 100 μg/mL tryptophan. A pair of matched cultures was established for each strain by dispensing 50 mL aliquots of the resuspended cells into different 500 mL glass Erlenmyer flasks with Teflon® lined screw caps. An inert organic carrier phase (10 mL Perfluoro-compound FC-75 (Fisher Scientific, Philadelphia, Pa.) was added to each culture. The carrier phase for one culture from each pair was supplemented with 24 mg of 2,6-DMN. The remaining cultures were used as unsupplemented controls. All of the cultures were incubated at 37° C. with reciprocal shaking. After 24 h of incubation, 1 mL of 20% glycerol was added to each culture. Samples (1.0 mL) of the aqueous phase were periodically removed from the cultures. The samples were centrifuged to remove bacteria. The sample supernatants were passed through 0.22 μm Acrodisc® CR PFTE filters and analyzed for metabolites of 2,6-DMN by HPLC. The final sample (10 to 12 mL) was acidified to pH 2.0 using hydrochloric acid and extracted with an equal volume of ethylacetate. The extract was treated with anhydrous sodium sulfate to remove residual water and was then evaporated to dryness according to standard protocols familier to those skilled in the art. The dried residues were derivatized with 1 mL BSTFA (bis(trimethylsilyl)trifluroacetamide silylation reagent (Supelco, Bellefonte, Pa.)) according to standard protocols and analyzed by GC/MS.

Two 2,6-DMN metabolites were detected by HPLC in cultures of Clone 4a and Clone 6f after 120 h of incubation when 2,6-DMN was present in the organic carrier phase. The metabolites were presumptively identified by comparing the retention times (RT) of the metabolites to the retention times of authentic 6-M-2-HMN (RT=22.041 min) and 6-M-2-NA (RT=14.262 min). Production of 6-M-2-HMN and 6-M-2-NA by Clone 4a and Clone 6f was confirmed by GC/MS. The mass spectra for both metabolites were identical to the mass spectra of the corresponding standards. The 2,6-DMN metabolites were not detected in the culture of TOP10(pCR®2.1-TOPO™) that contained 2,6-DMN (data not shown). Furthermore, neither 2,6-DMN metabolite was detected in the cultures of TOP10(pCR®2.1-TOPO™), Clone 4a and Clone 6f that lacked 2,6-DMN (data not shown).

In addition to the two metabolites that were originally detected in by HPLC, production of additional metabolites by Clone 6f (metabolite 3) and Clone 4a (metabolite 4) was detected by GC/MS after 288 h of incubation. The mass spectrum of metabolite 3 produced by Clone 6f matched the mass spectrum of authentic 2,6-HMN. The mass spectrum of metabolite 4 produced by Clone 4a matched the mass spectrum of authentic 2,6-NDC.

What is claimed is:

1. A process for the oxidation of a substituted polycyclic aromatic substrate comprising:
(i) providing a recombinant microorganism comprising a DNA fragment encoding a xylene monooxygenase enzyme comprising an xylA subunit and an xylM subunit;
(ii) contacting the recombinant microorganism of step (i) with an aromatic substrate according to formula I 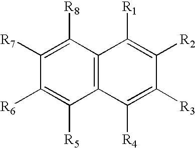
wherein R1-R8 are independently H, or CH3, or C₁ to C₂₀ substituted or unsubstituted alkyl or substituted or unsubstituted alkenyl or substituted or unsubstituted alkylidene, and wherein at least two of R1-R8 are present and are not H;
(iii) culturing the microorganism of step (ii) under conditions whereby anyone or all of R1-R8 is oxidized.

2. A process for the in vitro oxidation of a substituted polycyclic aromatic substrate comprising:
(i) providing a xylene monooxygenase enzyme comprising an xylA subunit and an xylM subunit;
(ii) contacting the enzyme of step (i) in vitro with an aromatic substrate according to formula I 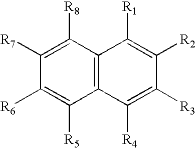
wherein R1-R8 are independently H, or CH3, or C₁ to C₂₀ substituted or unsubstituted alkyl or substituted or unsubstituted alkenyl or substituted or unsubstituted alkylidene, and wherein at least two of R1-R8 are present and are not H;
wherein anyone or all of R1-R8 is oxidized.

3. A process according to claims 1 or 2 wherein the aromatic substrate is selected from the group consisting of 2,6-dimethylnaphthalene, 1,2- dimethylnaphthalene, 1,3-dimethylnaphthalene, 1,4- dimethylnaphthalene, 1,5-dimethylnaphthalene, 1,6- dimethylnaphthalene, 1,7-dimethylnaphthalene, 1,8- dimethylnaphthalene, 2,3-dimethylnaphthalene, 2,4- dimethylnaphthalene, 2,5-dimethylnaphthalene, 2,7- dimethylnaphthalene 2,8- dimethylnaphthalene; 6-methyl-2-hydroxymethylnaphthalene, 6-methyl-2-naphthoic acid, and 2,6-bis(hydroxymethyl)naphthalene.

4. A process for the production of 2,6-naphthalenedicarboxylic acid comprising:
(i) providing a recombinant microorganism comprising a DNA fragment encoding a xylene monooxygenase enzyme comprising an xylA subunit and an xylM subunit;
(ii) contacting the recombinant microorganism of step (i) with an aromatic substrate selected from the group consisting of 2,6-dimethylnaphthalene, 6-methyl-2-hydroxymethylnaphthalene, 6-methyl-2-naphthoic acid, and 2,6-bis(hydroxymethyl)naphthalene; and
(iii) culturing the microorganism of step (ii) under conditions whereby 2,6-naphthalenedicarboxylic acid is produced.

5. A process for the production of 6-methyl-2-hydroxymethylnaphthalene comprising:
(i) providing a recombinant microorganism comprising a DNA molecule encoding a xylene monooxygenase enzyme comprising an xylA subunit and an xylM subunit;
(ii) contacting the recombinant microorganism of step (i) with 2,6-dimethylnaphthalene; and
(iii) culturing the microorganism of step (ii) under conditions whereby 6-methyl-2-hydroxymethylnaphthalene is produced.

6. A process for the production of 6-methyl-2-naphthoic acid comprising:
(i) providing a recombinant microorganism comprising a DNA molecule encoding a xylene monooxygenase enzyme comprising an xylA subunit and an xylM subunit;
(ii) contacting the recombinant microorganism of step (i) with an aromatic substrate selected from the group consisting of 2,6-dimethylnaphthalene and 6-methyl-2-hydroxymethylnaphthalene; and
(iii) culturing the microorganism of step (ii) under conditions whereby 6-methyl-2-naphthoic acid is produced.

7. A process for the production of 2,6-bis(hydroxymethyl)naphthalene acid comprising:
(i) providing a recombinant microorganism comprising a DNA molecule encoding a xylene monooxygenase enzyme comprising an xylA subunit and an xylM subunit;
(ii) contacting the recombinant microorganism of step (i) with an aromatic substrate selected from the group consisting of 2,6-dimethylnaphthalene and 6-methyl-2-hydroxymethylnaphthalene; and
(iii) culturing the microorganism of step (ii) under conditions whereby 2,6-bis(hydroxymethyl)naphthalene acid is produced.

8. A process according to any of claims 1, or 4-7 wherein the culturing of step (iii) occurs in a medium comprised of culture medium for bacterial cell growth and an organic solvent for delivery of the organic substrate.

9. A process according to any of claims 1, or 4-7 wherein the recombinant organism is selected from the group consisting of bacteria, fungal and yeast species.

10. A process according to claim 9 wherein the recombinant organism is selected from the group consisting of Aspergillus, Trichoderma, Saccharomyces, Pichia, Candida, Hansenula, Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium, Klebsiella, Burkholderia, Novosphingobium, Sphingomonas, Paracoccus, Pandoraea, Delftia and Comamonas.

11. A process according to claim 10 wherein the recombinant organism is Escherichia coli.

12. A process according to any one of claims 1-7 wherein the xylene monooxygenase enzyme is isolated from a member of the Proteobacteria.

13. A process according to claim 12 wherein the member of the Proteobacteria is selected from the group consisting of Burkholderia, Alcaligenes, Pseudomonas, Novosphingobium, Sphingomonas, Pandoraea, Delftia and Comamonas.

14. A process according to any of claims 1-7 wherein the xylM subunit is encoded by an isolated nucleic acid selected from the group consisting of:
(i) an isolated nucleic acid molecule encoding the amino acid sequence selected from the group consisting of SEQ ID NO:10, SEQ ID NO:16 and SEQ ID NO:20;
(ii) an isolated nucleic acid molecule having 95% identity to (i); and
(iii) an isolated nucleic acid molecule that is completely complementary to (i) or (ii).

15. A process according to claims 1-7 wherein the xylA is encoded by an isolated nucleic acid selected from the group consisting of:
(i) an isolated nucleic acid molecule encoding the amino acid sequence selected from the group consisting of SEQ ID NO:12, SEQ ID NO:18, and SEQ ID NO:22;
(ii) an isolated nucleic acid molecule having 95% identity to (i); and
(iii) an isolated nucleic acid molecule that is completely complementary to (i) or (ii).

16. A method for identifying a nucleic acid molecule encoding a xylene monooxygenase comprising:
(i) probing a genomic library with a portion of a nucleic acid molecule selected from the group consisting of SEQ ID NO:9, 11, 15,17, 19, and 21;
(ii) identifying a DNA clone that hybridizes under conditions of 0.1×SSC, 0.1% SDS, 65° C. and washed with 2×SSC, 0.1% SDS followed by 0.1×SSC, 0.1% SDS with the nucleic acid molecule of (i); and
(iii) sequencing the genomic fragment that comprises the clone identified in step (ii),
wherein the sequenced genomic fragment encodes xylene monooxygenase.

17. A method for identifying a nucleic acid molecule encoding a xylene monooxygenase comprising:
(i) synthesizing at least one oligonucleotide primer corresponding to a portion of the sequence selected from the group consisting of SEQ ID NO:9, 11, 15, 17, 19, and 21; and
(ii) amplifying an insert present in a cloning vector using the oligonucleotide primer of step (i);
wherein the amplified insert encodes a xylene monooxygenase.


SEQUENCE LISTING (22 sequences)

SEQ ID NO: 1 - DNA, 17 bp, Artificial Sequence
  misc_feature: primer
ccgcacgattgcaaggt

SEQ ID NO: 2 - DNA, 17 bp, Artificial Sequence
  misc_feature: primer
ggtgggccacacagata

SEQ ID NO: 3 - DNA, 15 bp, Artificial Sequence
  misc_feature: primer
acgggcggtgtgtac

SEQ ID NO: 4 - DNA, 16 bp, Artificial Sequence
  misc_feature: primer
gccagcagccgcggta

SEQ ID NO: 5 - DNA, 856 bp, Sphingomonas sp.
ggaaggagctagcgttgttcggaattactgggcgtaaagcgcacgtaggcggcgatttaagtcagaggtgaaagcccggggctcaaccccggaactgcctttgagactggattgctagaatcttggagagacgagtggaattccgagtgtagaggtgaaattcgtagatattcggaagaacaccagtggcgaaggcggctcgctggacaagtattgacgctgaggtgcgaaagcgtggggagcaaacaggattagataccctggtagtccacgccgtaaacgatgataactagctgccggggcacatggtgtttcggtggcgcagctaacgcattaagttatccgcctggggagtacggtcgcaagattaaaactcaaaggaattgacgggggcctgcacaagcggtggagcatgtggtttaattcgaagcaacgcgcagaaccttaccagcgtttgacatcctcatcgcggatttcagagatgatttccttcagttcggctggatgagtgacaggtgctgcatggctgtcgtcagctcgtgtcgtgagatgttgggttaagtcccgcaacgagcgcaaccctcgcctttagttgccagcatttagttgggtactctaaaggaaccgccggtgataagccggaggaaggtggggatgacgtcaagtcctcatggcccttacgcgctgggctacacacgtgctacaatggcgactacagtggcgtgcaaccgtgcgagcggtagctaatctccaaaagtcgtctcagttcggattgttctctgcaactcgagagcatgaaggcggaatcgctagtaatcgcggatcagcatgccgcggtgaatacgttcccaggcct

SEQ ID NO: 6 - DNA, 12591 bp, Sphingomonas sp.
ctcggtacccccagcgcaatctccggcttggtgcggtagtgccggaaatcctcgggaacgccggcacgttccaggcgagccatgtcgcttgtccaactttccggcaggaacaggcgcaagcccaccatgatcggtacctcgcccgaagccaacgtcagcgacaccagcgtctggcagttggcgttcttgccgagggctgaggcatattgcggcgccacgccgaccgaatgacgccccttcttgggcaaggccgtatcgtctatgatcagccaggcatcatcaccacccacttgccggttagcttcggcaagcagcgccatctccagtggagcttcgtcccagacgccgctaccgatgaagtgatgcagcttgtcatagctgaccccgtcatctcgggctgccatcggctgcacgctcttgcggtcgcctggaccaatcagacccgctatgtaggcagggcacatccgaccgcgcgtcttgttccgcagagctcccacaaacggtgaaagccacgcgtccagttcgctccgccaatccccatgcatcgccagcccccacagaaactggctctctatgaatcaccggaatcctgacttgggaatccaaaaaatcatatttctgccaaagtagtgctaggtttattcctggatttccaacttttcaagacgcaaggcgagctgccgacgggtaaagcctaaggtgcgcgccgcgcgcgcaacgttgcccccactcgcatcaagggcctcgcggatcaccagcccttccatctccagcaggcttccgcccccatcgacgatcagatccagcatctggcgcaccaagccggcgcgatcgccgacattgtcaacggcgctgatcctgccatcgttggtcatgcccatcgccggcttcaattcgagaccgtcggtttcgaggaacaggtgcctgacgtcgatcgctcgcccgtcgtccgccagcagcacggcgcgttccaccattcgctcaagttcgcgcacattgccggggaaatcgtacaccagcagcgcgttgatcgcctgcggggtgaagcccgataccgttcggccatggcgcatggcataacgcagacggaagtgttccagcagcaccggaatgtcctcgccgcgctggcgcagcggcggcacccggaccggcaaagtcgatatccggtagaacaagtcggcacgaaacgtgccttcccgcaccgcctcgttaagttccacattggatgctgcgatcaatcggacatccaccttgatcgtccgcgtatcgccgacccgctcgaactcaccttcctggacagcgcgcagaatcttcgattgtgcaagtggcgacagtgtcgaaatctcgtccaggaacagcgtgccgccattggccagctcgaaccgaccgggccgggcggcaaccgcgccagtataggcccccttggccacgccgaacagctccgattcgatcaacccctcggggatcgccgcgcagttgagcgcgatgaacggctccgcctcgcgcgtgctcagccgatgagcgagcttggccagcacttccttgccgaccccggtctcgcccattaacagcagcgtcgcattgctcgacgcggtgcgttcgatcatggtcttggctgagaggaaccctgccgagatcccaatcaccgtttggcccccatcatgccacgtttcggacaggaccagcggcgtcgcggccaattcgaggttgtccaactgatcccattcgggttttggtttggcgtgaagtacgcaggcgacatcgcctcgccccgcacattccacttcgcgcatgacgatcggttgtcccgcgaaggttgtggcaaagccgctggcaaagccggtctgcatccagcacaccggatcggtgctgtgcccgatcgcgcttagatggatcgccgcctcgatcgaatcgtggaccctgaaccggccttcgaaatgcccccgcgagggatcattttccagggtttcgatctgggtccagccgaacccggtcaaggcgtgcgcctgcggtccgaccgcgaatgcttcgagataatcaccatccgggcgcaatttcttcgcgccgatcgcgcaccttgcgccttcggcaaaaccgacaccccagaagaaccggctggccgcttcgtgccccaattcggcaaccaggcagctgcgccagctggccagagccgcttggctgagcagtacgcagcgttcaccatcgtgccagatccgggccatttccggtgagaactgcagcttgctggcaatgtcggcgtagcctggaagtgtcttcacccggtcaggctatcagattcgtaactttcgggccagactcaagcctcgacatttacatttacgtcgatctgcaaggtgcctcgcagtgggattgaccagcccggactgctgcaaaccagttagaaagcgatattggcacgattcgtgcttaaaatttcccggcatggcaaaccgatgccggctttagccatggtgtgaggagaggatcaaatgaacgacagcattgccgatctggttgattctcgcaccgggcgccaatcgcgctcgatctacgcgagcgaagacatctatcggcaggaacttgagcggatcttcgggcgctgctggctgttcctggtccacaccagccagattccgaagccgggcgactatttccgcaccttcatgggcgaagacgatgtgatcgtgatccgccagaaggacgggtcgatcaaggcgttcctcaacagctgtacccatcgcggcaaccggatctgccgcgccgatcgcggcaatgcgcgcgctttcacctgcaactatcacggctggtctttttccccggacggcgcgctctccggggtgccgctggaaaacgaggcctatttcggcgaactcgaccgcaccaagttcggcctgatcccggtgacgaaagtggccgagtataagggcctggtgttcggctgctgggatgccaattcgcccagcctcgatgactatctgggcgatgccaagtttttcctcgatgtctggctggatgccatgccaggcggatcggcactgctcggcgagacgcagaagatggtgctgggcaccaactggaagctgccagtcgagaacgtctgcggcgatggctatcacctgggctgggcccatgctggcgcgatggcggcggtccagtcgatggacctcaccgggctcagcgtcggcaattccggggtcgatctcgatggcgggctgtcggtcgccggcatgaacgggcacatggtcctgagcgcgctcgacggcgtttccggctatgccttctatcccgatcccaagccgatcctcgaatacctggaggccaaccgccagacggtgatcgaccgtctgggcgaagtgcgcggcaggcaggtgtggggtgcgcaggtcaacatcaccattttccccaacctgcaactgctgcccgggctcaactggttccgggtctatcatcccaagggtcccggccagatcgagcagtggacttgggccatggccgaaaacgacatgcccgaggcggtgaaagcgcagatcctggaaaaccagtgcctgacattcggcctggcgggcctgttcgacaacgacgatggcgacaatctgaccgctgcaccgaacagtcgcgcggctggcgcacggcgcacatggatgtctacaccaacatggcggtgggccgctcgggcaagcgcgagggcttccccggcgatatcgccgccggcttggtaagcgaacacaaccagcgctatttctaccgccgctggcaagagcacatgatggcggaaacttggccgaagtgcccacgtacaacatcaactcgttgaccgaacaggaagccgagcatgcttgaccggccgcttgcccctcccccggtggaggtgatcgttgctgttacccaggcactccaccgcgaagcccggctgctcgacaacgaagatttcgaaggctggctggccatgctcggcaaagacgtcagctatcgcctggaactgaagagccgaaggttccgcgctgaccgttcgccgcctttgccatttggcccaggggtaatcttcaacgaggatctgggtcggctgaaaatgcgggtggatcgcctgaaatccggcttcgtctgggccgaagatccgcccaactacatccgccgcgctgtctccaacgtcgaggtgctggccacccgggccgataacgaggtgcgggtccattcggtgctggggatgcaccgcaatcggatcgatggcaccacccgcctgctcaccgccggacgcactgacctctggcgcagcgaaggcagcgcttggctgctggccgcgcgcgaaatcgtgctggaccattccgtgctgccggacagcaacctcaacgcctttttctgaacaagcctgacgggagagaacaatcatgcgcttcgaacggatcggtcgcgaaccggattattcacgctacatggacctcaaggaaggctggcttgaccgccggatcttttcggatgcggacatctacgaggaggagctgtaccgcatcttcgcgcggtcgtggctgttcgttgcccacgaaagccagatcccaaacagcggggacttcctgacgacccacatgggtgaagacgcggtgatcgtcgcgcgccagcccgacggttcgatccgggtcatgctcaattcctgcccgcaccgcggcaataaggtgtgcttcgccgatgccgggaacacccgtcggttcgtctgcaattaccacggctgggcgtttgacaccgccggcgacctcaagggcatgcacgaggaatattgctacgacgcgggcgatatcgacttcaagaaccatggcctcaagaacgtcgccaaggtcggcaactacaagggcttggtgttcgccaccttcaacagcgatgcgccgagcctggaagcctggctaggcgatttccggtggtatctcgacatgatcctcgacaacgaggaaggcggcaccgaattcattggcggctgcatcaagtcggtgatcagcgcgaactggaagttcggggtcgagaacttcatcggcgacgcttatcacgccggctggacgcatgattcgggcactcggtcgatgaacaacggccagccgttcccgccgatcgacatggataattcctatcacgccagcgtgaacggccacggctgggaattcggcacccaaggcgtgggcgacctcttcctgctcgggcgccccaaggtgatggactattacaacaagatccgcccgaagatggcagaacgcctgggcgagatgcgctcgaagatcttcggttcggtcgcctcggcatcgatcttccccaacgtctcgttcctgccgggcatttccaccttccgccagtggcaacccaaggggccaatgcagttcgaattgaagacctgggtgatcgtcaacaagaacatgcccgacgacatcaaggaggaagtgaccaaaggcgtgatgcagacctttggccccggtggcaccttcgagatggatgacggcgaaaactgggaaaactgcaccaccgtcaaccgcggcgtcgtcacccggcacgagcgcctgcactatcgctgcgggatcggccgccagatcgaacacgataccctgccgggcttcgtctatcgcggccagtacaacgacgccaaccagcgcggcttctatcagcgctggctcgacatgatgacccatgacgaattcggcaagatgccggcacggcccgaaccgcagctgggcaatgtgggcgaaacccgcgatcttcctggcctgttcgcgctctgagaggaactacagctatgaccgacaccgcaaccctcgagcgcacatgtgaagggactaccgcatcgctcgaactgacccacgcgctgacccagacgctgtatcgcgaagcacggctgctcgacgatgaacactacaaggcctgggccagcatgctggcggaagacctgcattaccacatgccggggatcgaaacccgctatcgccgcgacaagaccgagcaggtgaccgacctgacccggatggcctattacaacgacagcaagcccgaaatcctcaagcggctttctcggctcgagaccggaacggcctggtcggaggatccggcgacgcgctacacccatatcatcaccaacgtcgaggtggagcttaccgaaaaggccgacgagttccgagtctattcgaacttctatgcttatcgcaatcgcaacgaacgcgacgaggattcgctaatcggcaaccgtatcgatatctggcgtcaggtcggcaacagctttcaactggtcaaacgccgagcgatcctcaagcacaatgtaatgttgagcaagaacctcaacatctacgtttagtctatccacctttataaatagtaatgataaaacacgagggagaatgccaaatgacaaaccgattgatgtatttcaggaaagccgtcctgcgcgccagcgcctcggcgatcgtagtatctaccgggcttgtgccgatgacggcgcatgcgcaggatgcagcagcaagcgagcctcaggccgaggagcaacgggccggcggtctaggtgaaatcatcgtgactgcgcgcaagcggtctgagaacctgcaggaaacgccggtcgcgatcaccgctatgaatgccgagatgctcgaagcgcgcgaagtcaacaacgtcgcccaggtggccaagttcgcgcccaacgtgaacatgtcgccggttgccaacatctccggttccagcgccacgatcaccgcgttcatccgcggcgtcggacagaccgacttcaacattaccgtggataccggcgtgggcatctatgtcgatggcgtctatgtcgcccgctcggtcggtgcgctgctcgacatgaccgacatcgctgatgtgcagatcctgcgcggccgcagggcacgttgttcggcaagaacaccatcggcggcgcgatcgtcgtcaattccgtccagccgcaacacgaatttgacctgaagctcgaagcggcgaccggccggttcaaccgcgccgatttcaagggcatgatcaacgtgccgctgagcgacaatctcgcaatgcgcgcggttgcttcctatgaaacccgcgacggctaccagaagcgtttgttcgacggcggccgccagggcaacaaggacagcttcggcggacgcattgcgttcaagtgggagccgaccgataagctgaccgtcagcctcagcggggatatcaacatccgccgcgaagagcagacggccatttcgctgctcgaactgcaggaccagaacgtgccgctgcgcttcgtcgatatcccgaacagcgcgacgacacccggcggccccaaccgccagaccgctgcgcccagctcgatgtatttctggaacaagatccgcgtcaccgatggcagctgtggagcaccttggggcgggtttggcgttccgggtacccttgcgccgaccggcaacccggcctgcgccagcacccgctggatcactggcgacatcgacaccacctgggctggcggcgtcaaccggtcggacttcgatctatggggcacgaacctcaccctcgattacgatttcggcgatttcagcctgaagtcgatttcggcctaccgcgaccagagttcgcacatggaatacgatttcgacggcactccgcatacgatcctgcggatcgcaagcgatatcgatgtgtggcaggcctcgcaggaactgcagtttaccggaagcctgatggatggccaggtgaagttcgttctgggcggctattatctcaaggaaaagggcactgactacaccccgatggaattcgggtttgcccagttctttactggcggcgacatcgacaacgacagctatgcctcatacctgcaggccaccttcaaggtgaccgaccggttctcgatcacgccgggcatccgctacactaacgagaccaagcggttcgacccgtctgtgcagacgatcttcaacgaccgctcgcaactcgatccagtgctggcatcggtctatccgcagggcgcattcgtcgcgttcagccagtgcctcgtcggacaggcaaacccggcggccaacctgttcccgtcaggcccgctggccggtttcccgctaccgaactgcacgccttcggcgaccaacccggcggcaaccacacgatgccggccatcgaggtttcggccaaggccaaggagtggaccccggcgatctcggcggactacaagataaccgacaattcgctgatctatgcgtcctattcgaaaggcttcaagaacggcgggttcagccagcgcatcttcccggctgaaatcgcgaccccctcgttcacgccggaattcgtcgaatcctacgaaatcggcctgaagaacgaactgttcaaccggcgcctgcgcctgaacatcgcggcgttcctttccgattacagcgacatgcagatcactgtgaacgaagggatcgctccgaaggttcgcaacgccggtgccggccggatcaagggcttcgagatcgaaggcgaagctgccccgatcgaacaggttcgcgtgaacttcggcgtgggttatctcgatgcctactacaccaagatcgatccgagcgcggcgccagtcacgctggattcgaagttcgccttcgttcccaagtggacagcttcggccgcgatcaacgccgacgtctatgaaggcagcatgggcaagctgaccctgcgcggcgattggtcctaccagagcggcaccttcaaggatgcagtcaacagcccgcaactgttccagcccgcgtacagcgtgttcggcaccagtgcctcgttcaccgacaggagtgagcacttcaccctgactgcgggcgtcacgaacctgaccgacaagcgctatctgcagggcggctatgtcgatctcaacgtcggcggcgccgcaacggccagcttctcgcgtccgcgcgaatggttcctcaagctcgcctacaagtactgatgaaggcccgagggcggcggaccgccaccgccctcacccttgcctcgacgggcaaggccagaacagaataactaaggagaaatccgatggacggactgcgctattttctcattccggtcatgaccttggcgggcgttatcggattcatgctcggcggcagctacgtctggctgggcgcggcgacctttccggtgttgatgacgctggatatcctgctgccggcggaccacaagatgcgcgcgcagggcacggcactgcttggcgatttcgccatgtacctccagctcccgtgcatgatcctgctggtctgggcctttgcccgatcggttgcgaccgcgatcaacccgatcaccggcgccgacaattcaagctggcaactggccggatcgctgctcagcctgggttggctctcggcggtgccgaccctgccggtggcgcatgagctgatgcaccgccgccactggtttcctcgctatgtcgccaagtgtctgagcgcgttctacggcgaccccaatcgcgatattgcccatatcgtcacccaccacgtccatctcgacacggccaaggatagcgatacccctcggcgcgggcagaccatctacagcttcgtgttccaggcgagttggggttcctacaaggatacttgggaaaagtcggccgaaatcctccgcaagcttggccatgcttcgctgggatggcgcaatccggtgtggctactgccgctcttatcgggcagtatcatcgtctttgtggcttttacggcaggccttggcgcggcgctgaccgctgtcggcgcgatggtaatggccaagatgttcgtggaggggttcaactacttccagcactatggcctgatccgggtcgaaggggcgccgatcgagctccatcacgcctggaaccaccttggcgcgatcgtccggccgatcggtgcggaaatcaccaaccacatcaaccatcaccttgacgggcatatccccttctatgcgctgaagcccgaaccgcaagcgccgcagatgccctcactgttcctgtgcttcgccgccggactgatcccgccggtctggttccgcttcatcgcgcaaccgcgcctcaaggactgggacgaacgtttcgccacccccggcgaacgcaagctggcggatcaggccaacgcacaggctggctggccgcgctggctggccagcacttgaacgccaggccaatcgtccaccctcattcgggcgacaatctgcaactgcagattccgcccgcccatttcggaaaaacccgacaatgttttctttcctgcgcaaatccaagatgaacaccgtaaccgtggaaggatcgccgaccacgcttgacataccggcgggcaagacacttcttgaagcgatgctggacgcgggtctggccatgccgcacgattgcaaggttggctcgtgcggtacctgcaagttcaagctcgtgtctggcaagatcggcgaattgagcccgtcggcccttgcacttgagggcgacgaactgcgcagcggctttcgcctcgcctgccaggccattccgcgctcggatctgacaatcgcggttgatgcgccactctcgcaagggatcgccattgctacatatcgcggcaccatcgtcgccgcacaacggctatgcgaggatataatcgggctgaccatcgaactggatcggccactggccttcactcccggacaatacgctgatctgaccgctcccggcatcgaaggtgcacgcagttattcattcgccttcgcgacggttggtgaacccacccagcaactgcattttcacatccggcacgttccgggcggcgcatttaccgactggctgttctgcaccgatcgcaccggaatggagctgaaggttaccgccccctatgggcaattcgccctcaaggacagcactgcccccattctttgcatcgccgggggtagtggtctagcgccgatcatctcgattctggagcaggcgctcgaccggggcgcagaccgggcggtgcacctgctgtacggtgcgcgccgtaagtccaatctctatgccctcgacaaaattgcggcccttcgtcaacgctggatggcccctttcgaattcgtcccggccttgtcggatgaagagccagacagcgactgggcaggagcgcgtgggctgatcaccgagcagattgcgggcgttgcagacctggcggcgcacgaagcctatctgtgtggcccaccggcgatgatcgactttgccgaagcgcaattgcttgccgccggcctctcacgttcggtcatttcggccgaccgctttcttgatcgcagcaatcgcacgtaaaaaaagacgggcgctcgatgaatcgagcacccgccaagggtggagagatctaagccgcttcagcttccggggcgtttgccgatgccgattccaccgtcgctgagcaaaaccgttcctgtcatgtaggccgagtcccgacgcgaggcgagcaaggcatagaggccggagtgatcatccggttcggcgatccgcgcgagcggcgtcatcccggcaatcatcgcgtcgagcccttccatatcttccatgtgaacatcggctgttccgcccgcatgggtaccgcccagcggcgttcgggtgccgcctggcgccacgccgttgaccctgacgtcgggggttagttcccaggcgagctggcggatcaggcccagcacggcgtgcttcgacgccacatagggcgtaccgccaccgccggtgtagaagcttgaagtcaaggcagtaaagatgatgctgcccttggtcttacgcaactccgggattgcggcacgggcgccaaggaagtagccctttacgttgacgccgaagatctcgtccagcgtttccgagagcttttcgggttccatgtccgcaagcggggtcatgaaatcccagatcccgacattgccgacgaacacgtcaagcttgccgaatgcagccacggttgcgacgactgccttttcgttgtcgacataattgcggacatcgccctcaatcacgactactgcgttgccatgccgcgtacgcaccaagtcggcctgagcggcatccctgaccaggacgccaaccttggcaccttcttcgataaagcgcgcgacgaccgcctcaccgatccctgtggcgccgccggttagcagcgccacttgtccttcgagccttgcagtcacatcgttcctcccgaagatcgcgggttctcgatcactgatcgaacatcccgacgcggccttcctgaaggttgaccacgaccgactttgtctgggtgtagtggttcagcacttcatggctgaattcacgcccgaagccgctctgcttgtaaccgcccagcggcatgttggccttaaggttgtagtagcggttgatccagaccgtgccggtttcaagctgacgcgcgatccggtgggcgcgggcaagatccttggtccacacgccgccggccaggccgtaggtggtggcattggcctgctgcatcatgtcggcttcgtcgttccagctgattacagtggtcaccggcccgaagatctcctcctgcgagatgcgcattgtgttcttcgcattggtagacaccgtcggattgatgaagctgccatccgccagatttgcagcatagaacgggtaccgccggtcaaaacttgggccccttcctcggttgcgacatgcaggtagctttctaccttgtc

SEQ ID NO: 7 - DNA, 32 bp, Artificial Sequence
  misc_feature: primer
taactaaggagaaatcatatggacggactgcg

SEQ ID NO: 8 - DNA, 35 bp, Artificial Sequence
  misc_feature: primer
ggatcccgggtctttttttacgtgcgattgctgcg

SEQ ID NO: 9 - DNA, 1101 bp, Sphingomonas sp.
atggacggactgcgctattttctcattccggtcatgaccttggcgggcgttatcggattcatgctcggcggcagctacgtctggctgggcgcggcgacctttccggtgttgatgacgctggatatcctgctgccggcggaccacaagatgcgcgcgcagggcacggcactgcttggcgatttcgccatgtacctccagctcccgtgcatgatcctgctggtctgggcctttgcccgatcggttgcgaccgcgatcaacccgatcaccggcgccgacaattcaagctggcaactggccggatcgctgctcagcctgggttggctctcggcggtgccgaccctgccggtggcgcatgagctgatgcaccgccgccactggtttcctcgctatgtcgccaagtgtctgagcgcgttctacggcgaccccaatcgcgatattgcccatatcgtcacccaccacgtccatctcgacacggccaaggatagcgatacccctcggcgcgggcagaccatctacagcttcgtgttccaggcgagttggggttcctacaaggatacttgggaaaagtcggccgaaatcctccgcaagcttggccatgcttcgctgggatggcgcaatccggtgtggctactgccgctcttatcgggcagtatcatcgtctttgtggcttttacggcaggccttggcgcggcgctgaccgctgtcggcgcgatggtaatggccaagatgttcgtggaggggttcaactacttccagcactatggcctgatccgggtcgaaggggcgccgatcgagctccatcacgcctggaaccaccttggcgcgatcgtccggccgatcggtgcggaaatcaccaaccacatcaaccatcaccttgacgggcatatccccttctatgcgctgaagcccgaaccgcaagcgccgcagatgccctcactgttcctgtgcttcgccgccggactgatcccgccggtctggttccgcttcatcgcgcaaccgcgcctcaaggactgggacgaacgtttcgccacccccggcgaacgcaagctggcggatcaggccaacgcacaggctggctggccgcgctggctggccagcact

SEQ ID NO: 10 - protein, 367 aa, Sphingomonas sp.
MDGLRYFLIPVMTLAGVIGFMLGGSYVWLGAATFPVLMTLDILLPADHKMRAQGTALLGDFAMYLQLPCMILLVWAFARSVATAINPITGADNSSWQLAGSLLSLGWLSAVPTLPVAHELMHRRHWFPRYVAKCLSAFYGDPNRDIAHIVTHHVHLDTAKDSDTPRRGQTIYSFVFQASWGSYKDTWEKSAEILRKLGHASLGWRNPVWLLPLLSGSIIVFVAFTAGLGAALTAVGAMVMAKMFVEGFNYFQHYGLIRVEGAPIELHHAWNHLGAIVRPIGAEITNHINHHLDGHIPFYALKPEPQAPQMPSLFLCFAAGLIPPVWFRFIAQPRLKDWDERFATPGERKLADQANAQAGWPRWLAST

SEQ ID NO: 11 - DNA, 1038 bp, Sphingomonas sp.
atgttttctttcctgcgcaaatccaagatgaacaccgtaaccgtggaaggatcgccgaccacgcttgacataccggcgggcaagacacttcttgaagcgatgctggacgcgggtctggccatgccgcacgattgcaaggttggctcgtgcggtacctgcaagttcaagctcgtgtctggcaagatcggcgaattgagcccgtcggcccttgcacttgagggcgacgaactgcgcagcggctttcgcctcgcctgccaggccattccgcgctcggatctgacaatcgcggttgatgcgccactctcgcaagggatcgccattgctacatatcgcggcaccatcgtcgccgcacaacggctatgcgaggatataatcgggctgaccatcgaactggatcggccactggccttcactcccggacaatacgctgatctgaccgctcccggcatcgaaggtgcacgcagttattcattcgccttcgcgacggttggtgaacccacccagcaactgcattttcacatccggcacgttccgggcggcgcatttaccgactggctgttctgcaccgatcgcaccggaatggagctgaaggttaccgccccctatgggcaattcgccctcaaggacagcactgcccccattctttgcatcgccgggggtagtggtctagcgccgatcatctcgattctggagcaggcgctcgaccggggcgcagaccgggcggtgcacctgctgtacggtgcgcgccgtaagtccaatctctatgccctcgacaaaattgcggcccttcgtcaacgctggatggcccctttcgaattcgtcccggccttgtcggatgaagagccagacagcgactgggcaggagcgcgtgggctgatcaccgagcagattgcgggcgttgcagacctggcggcgcacgaagcctatctgtgtggcccaccggcgatgatcgactttgccgaagcgcaattgcttgccgccggcctctcacgttcggtcatttcggccgaccgctttcttgatcgcagcaatcgcacg

SEQ ID NO: 12 - protein, 346 aa, Sphingomonas sp.
MFSFLRKSKMNTVTVEGSPTTLDIPAGKTLLEAMLDAGLAMPHDCKVGSCGTCKFKLVSGKIGELSPSALALEGDELRSGFRLACQAIPRSDLTIAVDAPLSQGIAIATYRGTIVAAQRLCEDIIGLTIELDRPLAFTPGQYADLTAPGIEGARSYSFAFATVGEPTQQLHFHIRHVPGGAFTDWLFCTDRTGMELKVTAPYGQFALKDSTAPILCIAGGSGLAPIISILEQALDRGADRAVHLLYGARRKSNLYALDKIAALRQRWMAPFEFVPALSDEEPDSDWAGARGLITEQIAGVADLAAHEAYLCGPPAMIDFAEAQLLAAGLSRSVISADRFLDRSNRT

SEQ ID NO: 13 - DNA, 23 bp, Artificial Sequence
  misc_feature: primer
taagtaggtggatatatggacac

SEQ ID NO: 14 - DNA, 27 bp, Artificial Sequence
  misc_feature: primer
ggatccctagactatgcatcgaaccac

SEQ ID NO: 15 - DNA, 1110 bp, Pseudomonas sp.
atggacacgcttcgttattacctgattcctgttgttactgcttgcgggctgatcggattttactatggtggctattgggtttggcttggggcggcaacattccctgcactgatggtgcttgatgtcattttaccgaaggatttttcggccagaaaggtaagtccctttttcgcagaccttacccagtatttgcagttaccattaatgatcggtctatatgggctccttgtcttcggagttgaaaacgggcgtatcgaacttagtgagccgttacaagtggcagggtgcattctttctttggcttggcttagtggtgtgccaactcttccggtttcgcatgagttgatgcatcgtcgccactggttgcctcggaaaatggcgcagctattggctatgttttatggtgatccgaaccgagacattgcccatgtcaacacgcatcacctttacttagatacgcctctcgatagcgatactccgtaccgtggtcagacaatttacagtttcgtgatcagtgcgacagttggttccgtcaaagatgcgataaagattgaggctgaaactttacgtagaaaaggacagtcaccgtggaatttgtccaacaaaacatatcaatatgtcgcacttctgctcgctctgcctggcttggtttcttatctgggcgggccagcattagggttggttacgattgcttcgatgattattgcgaaagggatagtcgagggttttaattactttcagcactatggtttagtacgcgatttagatcagcctatcctcctgcaccacgcgtggaatcatatgggaacaattgtgcgcccgctgggttgcgaaattactaaccatatcaatcatcatattgacggctatacacggttctatgagttgcgtccggaaaaagaagccccgcagatgccttcgctctttgtgtgtttccttctagggcttattccgcctctttggttcgctctcattgcaaaaccaaagttgagagactgggaccagcggtacgcaactccaggtgagcgcgaactggctatggctgcaaataaaaaagcgggatggccactgtggtgtgaaagtgaactgggtcgggtggctagcatttga

SEQ ID NO: 16 - protein, 369 aa, Pseudomonas sp.
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 17 - DNA, 1053 bp, Pseudomonas sp.
atgaatgagttttttaagaaaatctctggtttatttgtgccgcctccggaatctaccgtttcagtcagagggcaggggtttcagtttaaggtgccacgcgggcaaaccattctggaaagcgctctgcatcaaggaattgcctttccgcatgattgcaaagtcggatcttgtgggacatgtaaatataaactgatatctggcagggtcaatgagttgacctcttctgctatgggtctgagtggcgatctgtatcagtccggctatcgtttgggttgtcaatgcataccaaaagaagatctcgagatagagctagacacagtgctcgggcaggcgttagttccaatagaaacgagtgccttgattagtaagcagaaacggctggcgcacgatatagtcgagatggaagtagtgcccgataagcagatagccttctaccccggccagtatgcagatgtagaatgtgcagaatgctctgctgtaaggagttattctttttccgctccgccccaacctgacggctccctgagcttccatgttcgccttgtcccaggtggagttttcagtggttggctatttggtggcgatcgtacaggagcgacactaaccctgcgagcgccttatggacagttcgggctccatgagagcaatgccacgatggtctgcgtagccggcggaacggggcttgctccaattaaatgtgttttgcagagcatgacccaggcccagcgagagcgtgatgtgttgttgttctttggagctcgtcaacaacgtgacctatattgcctcgacgaaatagaagcgctgcaactcgattggggtgggcgcttcgagcttattccagttttgtccgaagagtcttctacgtcgtcatggaaagggaaacgtggcatggtaaccgagtattttaaggagtacctcactgggcagccttatgaaggatacctttgcgggccgccccctatggtggacgctgccgagaccgagctcgttcgacttggtgttgcgcgggaattagtgtttgcggaccgtttttataatagacctccttgctag

SEQ ID NO: 18 - protein, 350 aa, Pseudomonas sp.
MNEFFKKISGLFVPPPESTVSVRGQGFQFKVPRGQTILESALHQGIAFPHDCKVGSCGTCKYKLISGRVNELTSSAMGLSGDLYQSGYRLGCQCIPKEDLEIELDTVLGQALVPIETSALISKQKRLAHDIVEMEVVPDKQIAFYPGQYADVECAECSAVRSYSFSAPPQPDGSLSFHVRLVPGGVFSGWLFGGDRTGATLTLRAPYGQFGLHESNATMVCVAGGTGLAPIKCVLQSMTQAQRERDVLLFFGARQQRDLYCLDEIEALQLDWGGRFELIPVLSEESSTSSWKGKRGMVTEYFKEYLTGQPYEGYLCGPPPMVDAAETELVRLGVARELVFADRFYNRPPC

SEQ ID NO: 19 - DNA, 1104 bp, Sphingomonas sp.
atggacggcctacgctattttctcatcccagtcatgaccttggcgggcgttatcggattcatgctcggcggtagctacgtctggctgggcgcggcgacctttccggtattgatgacgctggatatcctgctgccggcggaccacaagatgcgcgcgcagggcacggcactgcttggcgatttcgccatgtacctccagctcccgtgcatgattctgctggtctgggcctttgcccgatcggttgcgaccgcgatcaacccgatcaccggcgccgacaattcaagctggcaactggccggatcgctgctcagcctgggctggctctcggcggtgccgaccctgccggtagcgcacgagctgatgcaccgccgccactggtttccacgctatgtcgccaagtgtctgagcgctttctacggcgaccccaatcgcgatatcgcccatatcgtcacccaccacgtccatctcgatacggccaaggatagcgatacccctcggcgcgggcagaccatctacagcttcgtgttccaggcgacttggggttcctacaaggatacttgggaaaagtcggccgaaatcctccgcaagctgggccatgcttcgctgggatggcgcaatccggtgtggctaatgccgctcttatcgggcagtatcatcgtctttgtgggcttgacggcaggccttggcgcgacgctgaccgctgtcgccgcgatggtaatggccaagatgttcgtggaggggttcaactacttccagcactatggcctgatccgggtcgaaggggcgccgatcgagctccatcacgcctggaaccaccttggcgcgatcgtccggccgatcggtgcggaaatcaccaaccacatcaaccatcaccttgacgggcatatccccttctatgcgctgaagcccgaaccgcaagcgccgcagatgccctcactgttcctgtgcttcgccgccggactgatcccgccggtctggttccgcttcatcgcgcaaccgcgcctcaaggactgggacgaacgtttcgccacccccggcgaacgcaagctggcggatcaggccaacgcacaggctggttggccgcgctggctggccagcacttga

SEQ ID NO: 20 - protein, 367 aa, Sphingomonas sp.
MDGLRYFLIPVMTLAGVIGFMLGGSYVWLGAATFPVLMTLDILLPADHKMRAQGTALLGDFAMYLQLPCMILLVWAFARSVATAINPITGADNSSWQLAGSLLSLGWLSAVPTLPVAHELMHRRHWFPRYVAKCLSAFYGDPNRDIAHIVTHHVHLDTAKDSDTPRRGQTIYSFVFQATWGSYKDTWEKSAEILRKLGHASLGWRNPVWLMPLLSGSIIVFVGLTAGLGATLTAVAAMVMAKMFVEGFNYFQHYGLIRVEGAPIELHHAWNHLGAIVRPIGAEITNHINHHLDGHIPFYALKPEPQAPQMPSLFLCFAAGLIPPVWFRFIAQPRLKDWDERFATPGERKLADQANAQAGWPRWLAST

SEQ ID NO: 21 - DNA, 1041 bp, Sphingomonas sp.
atgttttctttcctgcgcaaatccaagatgaacaccgtaaccgtggaaggatcgccgaccacgcttgacataccggcgggcaagacacttcttgaagcgatgctggacgcgggtttggccatgccgcacgattgcaaggttggctcgtgcggtacctgcaagttcaagctcgtgtctggcaagatcggcgaattgagcccgtcggcccttgcacttgagggcgacgaactgcgcagcggctttcgcctcgcctgccaggccattccgcgctcggatctgacaatcgcggttgatgcgccactctcgcaagggatcgccattgctacatatcgcggcaccatcgtcgctgcacaacggctgtgcgaggatataatcgggctgaccatcgaactggatcggccactggccttcactcccggacaatacgctgatctgaccgctcccggcatcgaaggtgcacgcagttattcattcgcgttcgcgacggttggcgaacccacccagcaactgcattttcacatccggcacgttccgggcggcgcatttaccgactggctgttctgcaccgatcgcaccggaatggagctgaaggttaccgccccctatgggcaattcgccctcaaggacagcactgcccccattctttgcatcgcagggggtagtggtctagcgccgataatctcgattctggagcaggcgcttgaccggggcgcagaccgggcggtgcacctgctgtacggtgcgcgccgtcagtccaatctctatgccctcgacaaaattgcggcccttcgtcaacgctggatggcccctttcgaatttgtcccggccttgtcggatgaagagccagacagcgactgggcaggagcgcgtgggctgatcaccgagcagattgcgggcgttgcagatctcgcggcgcacgaagcctatctgtgtggcccaccggcgatgatcgactttgccgaagcgcaattgctcgccgccggcatctcacgttcggtcatttcggctgaccgctttctcgatcgcagcaatcgcacgtaa

SEQ ID NO: 22 - protein, 346 aa, Sphingomonas sp.
MFSFLRKSKMNTVTVEGSPTTLDIPAGKTLLEAMLDAGLAMPHDCKVGSCGTCKFKLVSGKIGELSPSALALEGDELRSGFRLACQAIPRSDLTIAVDAPLSQGIAIATYRGTIVAAQRLCEDIIGLTIELDRPLAFTPGQYADLTAPGIEGARSYSFAFATVGEPTQQLHFHIRHVPGGAFTDWLFCTDRTGMELKVTAPYGQFALKDSTAPILCIAGGSGLAPIISILEQALDRGADRAVHLLYGARRQSNLYALDKIAALRQRWMAPFEFVPALSDEEPDSDWAGARGLITEQIAGVADLAAHEAYLCGPPAMIDFAEAQLLAAGISRSVISADRFLDRSNRT